SEQUENCE LISTING (13 sequences)

SEQ ID NO: 1 - DNA, 298 bp, Artificial Sequence
  TIP epitope string
ctagcgccgccaccatgacctttctgaccagcgagctgcctggctggctgcaggccaacagacacgtgaagcctaccgccgcctacgccgccgtgaagaactggatgacccagaccctggccgcctacagctacatccctagcgccgagaagatcgccgcctacaaccggaacatcgtgaacagactgctgggcgacgccgcctatcaggggcctggcgcccctcagggcccaggcgctccacagggacctggagccccttgatgaattctgcagatatccatcacactggcggccgc

SEQ ID NO: 2 - DNA, 1000 bp, Artificial Sequence
  TIPEGFP epitope string
gctagcgccgccaccatgacctttctgaccagcgagctgcctggctggctgcaggccaacagacacgtgaagcctaccgccgcctacgccgccgtgaagaactggatgacccagaccctggccgcctacagctacatccctagcgccgagaagatcgccgcctacaaccggaacatcgtgaacagactgctgggcgacgccgcctatcaggggcctggcgcccctcagggcccaggcgctccacagggacctggagcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgct

SEQ ID NO: 3 - DNA, 6096 bp, Artificial Sequence
  shuttle vector for MVA-TIP control vector
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcatctggaaacgggcatctccatttaagactagatgccacggggtttaaaatactaatcatgacattttgtagagcgtaattacttagtaaatccgccgtactaggttcatttcctcctcgtttggatctcacatcagaaattaaaataatcttagaaggatgcagttgttttttgatggatcgtagatattcctcatcaacgaaccgagtcactagagtcacatcacgcaatccatttaaaataggatcatgatggcggccgtcaattagcatccatttgatgatcactcctaaattatagaaatgatctctcaaataacgtatatgtgtaccgggagcagatcctatatacactacggtggcaccatctaatataccgtgtcgctgtaacttactaagaaaaaataattctcctagtaatagttttaactgtccttgatacggtagtttttttgcgacctcatttgcactttctggttcgtaatctaactcattatcaatttcctcaaaatacataaacggtttatctaacgacacaacatccatttttaagtattatattaaaatttaatcaatgtttatttttagttttttagataaaaaatataatattatgagtcgatgtaacactttctacacaccgattgatacatatcattacctcctattatttctatctcggtttcctcacccaatcgtttagaaaaggaagcctccttaaagcatttcatacacacagcagttagttttaccaccatttcagataatggaataagattcaaaatattattaaacggtttacgttgaaatgtcccatcgagtgcggctactataactatttttccttcgtttgccatacagatcctacgtactcgagataaaaagcggccttggccgaggcggccacgcgtaggcctagatctggccttaatggccggatccggtacctttatattccaaaaaaaaaaaataaaatttcaatttttacaaaaagaattcatcaaggggctccaggtccctgtggagcgcctgggccctgaggggcgccaggcccctgataggcggcgtcgcccagcagtctgttcacgatgttccggttgtaggcggcgatcttctcggcgctagggatgtagctgtaggcggccagggtctgggtcatccagttcttcacggcggcgtaggcggcggtaggcttcacgtgtctgttggcctgcagccagccaggcagctcgctggtcagaaaggtcatggtggcggcgctagcagatccgtcactgttctttatgattctacttccttaccgtgcaataaattagaatatattttctacttttacgagaaattaattattgtatttattatttatgggtgaaaaacttactataaaaagcgggtgggtttggaattagtgatcagtttatgtatatcgcaactaccgggcatatggctatcgacatcgagaacattacccacatgataagagattgtatcagtttcgtagtcttgagtattggtattactatatagtatatagatgtcgacctgcaggtcgacgaagttcctatactttctagagaataggaacttcgcagccaagctggaattcatccactttggataagaaatctgcatgataaatatattgatatcctaccacctattaaagtaccattatctaatagcaataagatagataaacaaatgttttttgatgaagttattacgtggataaatatatatcttcaggaaaagggtattatgttaccagatgatataagagaactcagagatgctattattccttaactagttacgtctctttaggtacttattttgatacgttacaagtaaaaaactatcaaatataaatggaatctgattctaatatagcgattgaagaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgaagttcctatactttctagagaataggaacttcaacaatgtctggaaagaactgtccttcatcgatacctatcacggagaaatctgtaattgattccaagacatcacatagtttagttgcttccaatgcttcaaaattattcttatcatgcgtccatagtcccgttccgtatctattatcgttagaatattttatagtcacgcatttatattgagctatttgataacgtctaactcgtctaattaattctgtacttttacctgaaaacatggggccgattatcaactgaatatgtccgccgttcatgatgacaataaagaattaattattgttcactttattcgactttaatatatccatcacgttagaaaatgcgatatcgcgacgaggatctatgtatctaacaggatctattgcggtggtagctagagctgattcttttttgaatcgcatcaaactaatcacaaagtcgaacaaatatcctttattaagtttgacccttccatctgtaacaatagggaccttgttaaacagttttttaaaatcttgagagtctgtgaattttgtcaattgtctgtattcctctgaaagagattcataacaatgacccacggcttctaatttattttttgattggatcaataataataacagaaagtctagatattgagtgatttgcaatatatcagataatgaagattcatcatcttgactagccaaatacttaaaaaatgaatcatcatctgcgaagaacatcgttaagagatactggttgtgatccatttatgagctcgcgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 4 - DNA, 6840 bp, Artificial Sequence
  shuttle vector for MVA-TIP-Raet1e vector
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcatctggaaacgggcatctccatttaagactagatgccacggggtttaaaatactaatcatgacattttgtagagcgtaattacttagtaaatccgccgtactaggttcatttcctcctcgtttggatctcacatcagaaattaaaataatcttagaaggatgcagttgttttttgatggatcgtagatattcctcatcaacgaaccgagtcactagagtcacatcacgcaatccatttaaaataggatcatgatggcggccgtcaattagcatccatttgatgatcactcctaaattatagaaatgatctctcaaataacgtatatgtgtaccgggagcagatcctatatacactacggtggcaccatctaatataccgtgtcgctgtaacttactaagaaaaaataattctcctagtaatagttttaactgtccttgatacggtagtttttttgcgacctcatttgcactttctggttcgtaatctaactcattatcaatttcctcaaaatacataaacggtttatctaacgacacaacatccatttttaagtattatattaaaatttaatcaatgtttatttttagttttttagataaaaaatataatattatgagtcgatgtaacactttctacacaccgattgatacatatcattacctcctattatttctatctcggtttcctcacccaatcgtttagaaaaggaagcctccttaaagcatttcatacacacagcagttagttttaccaccatttcagataatggaataagattcaaaatattattaaacggtttacgttgaaatgtcccatcgagtgcggctactataactatttttccttcgtttgccatacagatcctacgtactcgagataaaaagcggccttggccgaggcggccacgcgttcctcttcacatcgcaaatgcaaatgcaaataataaagatatgaagatgagtcccacagagataaatcctttcttagaagtagagtggctgggaggtggaagcggggaagttgatgtaagctgggtgatactgggggaccttgaggttgatcttggcttttccttggactgctttaagaattcatcaattttctgtctgcattggggtatgaagtatttcaattgttgcacaaggtccccatcatctttccatttattcatgataacccctgattcatcattagctgatctccagctcatattctctgtgtagaaggtgaagaagtaactgtcactgatgttgaattcccaggtggcactaggagtttggccctggctttgcggataaatcatggtgacttgtaaatgtgggtaaccattggtcttgtgagtgtccactttggtgttagacaccttgtccctcaactcctggcacaaatcgttcagaggttgtgtcaaacatttgccagtggcatttgccatcttccctgggtcacctgaagtcatggtcttgtttatgttatttaaatggaggatagttatttcatccactgagcacttcacgtcacaccagggaaggtctgctgaggtaggatccttgatggtcaagttgcacctaagagagtgtgcatcatccagcccgttggtgtatccatagctcagtagaattaacagcttctgaatcataaaatgatggcgcttggtcactgctgccttggccatggtttcttctgacggtacctttatattccaaaaaaaaaaaataaaatttcaatttttacaaaaagaattcatcaaggggctccaggtccctgtggagcgcctgggccctgaggggcgccaggcccctgataggcggcgtcgcccagcagtctgttcacgatgttccggttgtaggcggcgatcttctcggcgctagggatgtagctgtaggcggccagggtctgggtcatccagttcttcacggcggcgtaggcggcggtaggcttcacgtgtctgttggcctgcagccagccaggcagctcgctggtcagaaaggtcatggtggcggcgctagcagatccgtcactgttctttatgattctacttccttaccgtgcaataaattagaatatattttctacttttacgagaaattaattattgtatttattatttatgggtgaaaaacttactataaaaagcgggtgggtttggaattagtgatcagtttatgtatatcgcaactaccgggcatatggctatcgacatcgagaacattacccacatgataagagattgtatcagtttcgtagtcttgagtattggtattactatatagtatatagatgtcgacctgcaggtcgacgaagttcctatactttctagagaataggaacttcgcagccaagctggaattcatccactttggataagaaatctgcatgataaatatattgatatcctaccacctattaaagtaccattatctaatagcaataagatagataaacaaatgttttttgatgaagttattacgtggataaatatatatcttcaggaaaagggtattatgttaccagatgatataagagaactcagagatgctattattccttaactagttacgtctctttaggtacttattttgatacgttacaagtaaaaaactatcaaatataaatggaatctgattctaatatagcgattgaagaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgaagttcctatactttctagagaataggaacttcaacaatgtctggaaagaactgtccttcatcgatacctatcacggagaaatctgtaattgattccaagacatcacatagtttagttgcttccaatgcttcaaaattattcttatcatgcgtccatagtcccgttccgtatctattatcgttagaatattttatagtcacgcatttatattgagctatttgataacgtctaactcgtctaattaattctgtacttttacctgaaaacatggggccgattatcaactgaatatgtccgccgttcatgatgacaataaagaattaattattgttcactttattcgactttaatatatccatcacgttagaaaatgcgatatcgcgacgaggatctatgtatctaacaggatctattgcggtggtagctagagctgattcttttttgaatcgcatcaaactaatcacaaagtcgaacaaatatcctttattaagtttgacccttccatctgtaacaatagggaccttgttaaacagttttttaaaatcttgagagtctgtgaattttgtcaattgtctgtattcctctgaaagagattcataacaatgacccacggcttctaatttattttttgattggatcaataataataacagaaagtctagatattgagtgatttgcaatatatcagataatgaagattcatcatcttgactagccaaatacttaaaaaatgaatcatcatctgcgaagaacatcgttaagagatactggttgtgatccatttatgagctcgcgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 5 - DNA, 5955 bp, Artificial Sequence
  shuttle vector for DNA-TIP control vector
tgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccggatccggccattaaggccagatctaggcctacgcgtggccgcctcggccaaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccgccaccatgacctttctgaccagcgagctgcctggctggctgcaggccaacagacacgtgaagcctaccgccgcctacgccgccgtgaagaactggatgacccagaccctggccgcctacagctacatccctagcgccgagaagatcgccgcctacaaccggaacatcgtgaacagactgctgggcgacgccgcctatcaggggcctggcgcccctcagggcccaggcgctccacagggacctggagccccttgatgaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctga

SEQ ID NO: 6 - DNA, 6482 bp, Artificial Sequence
  shuttle vector for DNA-TIP-IL-7 vector
tgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccggatccaccggtaggaggggccaacatgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtatataaacaggacatgtagcaacaacctccaagaatctactggtgctagagtatccctcgaggggcccaagcttacgcgtggccgcctcggccaaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccgccaccatgacctttctgaccagcgagctgcctggctggctgcaggccaacagacacgtgaagcctaccgccgcctacgccgccgtgaagaactggatgacccagaccctggccgcctacagctacatccctagcgccgagaagatcgccgcctacaaccggaacatcgtgaacagactgctgggcgacgccgcctatcaggggcctggcgcccctcagggcccaggcgctccacagggacctggagccccttgatgaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctga

SEQ ID NO: 7 - DNA, 7242 bp, Artificial Sequence
  shuttle vector for adenovirus-GFPTIP control vector
ctttcctgcgttatcccctgattctgtggataaccgtattaccgctagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggctccaccatgggaaccaattcagtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgcgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccggatccggccattaaggccagatctaggcctacgcgtggccgcctcggccaaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccgccaccatgacctttctgaccagcgagctgcctggctggctgcaggccaacagacacgtgaagcctaccgccgcctacgccgccgtgaagaactggatgacccagaccctggccgcctacagctacatccctagcgccgagaagatcgccgcctacaaccggaacatcgtgaacagactgctgggcgacgccgcctatcaggggcctggcgcccctcagggcccaggcgctccacagggacctggagcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatcccgtcgagatatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctggtgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 8 - DNA, 7877 bp, Artificial Sequence
  shuttle vector for adenovirus-GFPTIP-CD70 vector
ctttcctgcgttatcccctgattctgtggataaccgtattaccgctagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggctccaccatgggaaccaattcagtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttggtaccggatccaccggtaggagggccatcatgattccggaggaaggtcgcccttgcccctgggttcgctggagcgggaccgcgttccagcgccaatggccatggctgctgctggtggtgtttattactgtgttttgctgttggtttcattgtagcggactactcagtaagcagcaacagaggctgctggagcaccctgagccgcacacagctgagttacagctgaatctcacagttcctcggaaggaccccacactgcgctggggagcaggcccagccttgggaaggtccttcacacacggaccagagctggaggagggccatctgcgtatccatcaagatggcctctacaggctgcatatccaggtgacactggccaactgctcttccccaggcagcaccctgcagcacagggccaccctggctgtgggcatctgctcccccgctgcgcacggcatcagcttgctgcgtgggcgctttggacaggactgtacagtggcattacagcgcctgacatacctggtccacggagatgtcctctgtaccaacctcaccctgcctctgctgccgtcccgcaacgctgatgagaccttctttggagttcagtggatatgcccttgaccacaactccaggatgacttgtgaagctagagtatccctcgaggggcccaagcttacgcgtggccgcctcggccaaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccgccaccatgacctttctgaccagcgagctgcctggctggctgcaggccaacagacacgtgaagcctaccgccgcctacgccgccgtgaagaactggatgacccagaccctggccgcctacagctacatccctagcgccgagaagatcgccgcctacaaccggaacatcgtgaacagactgctgggcgacgccgcctatcaggggcctggcgcccctcagggcccaggcgctccacagggacctggagcaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatcccgtcgagatatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctggtgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 9 - protein, 15 aa, Artificial Sequence
  Vector expressed peptide
IPYKDLTSSNYVVKD

SEQ ID NO: 10 - protein, 15 aa, Artificial Sequence
  Vector expressed peptide
INDKQGENEKYLPFL

SEQ ID NO: 11 - protein, 15 aa, Artificial Sequence
  Vector expressed peptide
TKPDSYPLFDGIFCS

SEQ ID NO: 12 - protein, 15 aa, Artificial Sequence
  Vector expressed peptide
YRSLKKQIEKNIFTF

SEQ ID NO: 13 - protein, 15 aa, Artificial Sequence
  Vector expressed peptide
DKIDLFKNPYDFEAI

(19) United States
(12) Patent Application Publication
(10) Pub. No.: US 2010/0322896 A1
(43) Pub. Date: Dec. 23, 2010

(54) MOLECULAR ADJUVANT

(75) Inventors: Adrian Vivian Sinton Hill, Oxford (GB); David Wyllie, Oxford (GB); Karen Colbjorn Larsen, Oxford (GB); Alexandra Jane Spencer, Oxford (GB); Matthew Cottingham, Oxford (GB); Sarah Gilbert, Oxford (GB)

Correspondence Address: FENWICK & WEST LLP, SILICON VALLEY CENTER, 801 CALIFORNIA STREET, MOUNTAIN VIEW, CA, 94041, US

(73) Assignee: ISIS Innovations Limited

(21) Appl. No.: 12/681,556

(22) PCT Filed: Oct. 7, 2008
(86) PCT No.: PCT/GB08/03366

§ 371 Date: Aug. 31, 2010

(30) Foreign Application Priority Data
Oct. 5, 2007 (GB) 0719509.2

(51) Int. Cl. A61K 39/295; A61K 39/00; A61P 35/00; A61P 31/12; A61K 39/275; A61K 39/235; A61K 38/20

(52) U.S. Cl. 424/85.2; 424/202.1; 424/277.1; 424/232.1; 424/233.1

(57) ABSTRACT

This invention relates to the use of a molecular adjuvant to generate an immune response in a host. In particular, the molecular adjuvant may be a TLR signalling pathway component, a co-stimulatory molecule, an NKG2D ligand, IL- or IL-15.

This invention relates to the use of a molecular adjuvant to generate an improved immune response in a host.

Over the last 10 to 15 years extensive research and development has been undertaken in the development of “vectored vaccines” which can be used as vaccine delivery systems. Vectored vaccines include DNA vectors and recombinant viral and bacterial vectors, which are engineered to express an antigen of interest.

Ideally recombinant viral and bacterial vectored vaccines are unable to replicate in the cells of the vaccine recipient, thereby enhancing safety of the vaccines. Viruses which may be used in the production of such vectored vaccines include human and non-human adenoviruses, vaccinia, modified vaccinia Ankara (MVA), other poxviruses, adenovirus associated viruses, flaviviruses, herpes viruses, alpha viruses, retroviral vectors, CMV vectors, reovirus vectors, Newcastle disease virus vectors, VSV vectors and other suitable viruses. Poxviruses have been proposed as good candidates for vectored vaccines as they show high species specificity; for example, avipox virus is unable to replicate in mammalian cells (Paoletti 1996 PNAS USA 93, 11349-11353). Protective immune responses induced by recombinant vaccinia viruses in small animals were first reported in the 1980s (Panicali and Paoletti 1982 PNAS USA 79, 4927-4931; Smith et al 1983 Nature 302, 490-495). Subsequently the highly attenuated recombinant vaccinia virus MVA (modified vaccinia virus Ankara) (Sutter and Moss 1992 PNAS USA 89, 10847-10851) and NYVAC (New York vaccinia) (Tartaglia et al 1992 Virology 188, 217-232) have been shown to have good immunogenicity. Due to an acquired replication defect MVA does not replicate in human cells, but is able to express recombinant genes. Similarly, NYVAC has been molecularly attenuated to prevent replication in human cells.

The aim of vectored vaccines is to activate cell-mediated or antibody-mediated immunity in a host organism against an antigen of interest. Preferably the cell-mediated immunity includes stimulation of a T cell response. This may be evidenced by a CD4 + and/or a CD8 + T cell response in a host organism following administration of the vectored vaccine. T cells are critical components of the immune system, and are involved in the control of intracellular pathogens. An intracellular stage is a feature of many pathogens including Plasmodium spp, M. tuberculosis, Leishmania and HIV. The T cell role extends beyond the control of infectious disease, for example some tumours express tumour-associated antigens which can be controlled by T cells targeting them. Vaccines designed to elicit T cell based protection against diseases, such as cancer and tumours, are under development (Hill, A. V 2006 Nat Rev Immunol, 6(1), 21-32; Sander, C and McShane, H 2007 Clin Exp Immunol, 147(3), 401-11; Johnston, M. I. and Fauci, A. S 2007 N Engl J Med, 356(20) 2073-81; Kedzierski, L et al 2006 Parasitology, 133 Suppl, S87-112; Xue, S. A and Stauss, H. J 2007 Cell Mol Immunol, 4(3), 173-84), but limited immunogenicity and protective efficacy of the vaccines remain a limitation.

Thus, whilst vectored vaccines offer a good basis for developing new vaccines, the immune response elicited by such vaccines when administered to an organism, typically a human, is often not sufficiently strong to provide protection against infection and/or disease related to the antigen encoded by the vector. Where the antigen is from a pathogen, the vectored vaccine may be intended to confer protection from infection and/or disease caused by the pathogen from which the antigen of interest is derived. Alternatively, the antigen may be derived from a particular cancer or disease and the vectored vaccine may be intended to confer protection or to treat that particular cancer or disease in the host organism.

A known method to enhance the immune response of an organism to an antigen in a vaccine is to use one or more adjuvants (or immune potentiators). Wherein the adjuvant increases the strength and/or duration of an immune response to an antigen relative to that elicited by the antigen alone.

Known adjuvant compositions include oil emulsions (Freund's adjuvant), oil based compounds (e.g. MF59, ISA51, ISA720), saponins, aluminium or calcium salts (i.e. Alum), non-ionic block polymer surfactants, lipopolysaccharides (LPS), attenuated or killed mycobacteria, tetanus toxoid, monophosphoryl lipid A, imiquimod, resiquimod, polyI:C, CpG containing oligonucleotides, lipoproteins and others.

Many adjuvants produce undesirable side effects in humans such as inflammation at the site of injection, these side effects can limit their use and efficacy, and thus there is a need for alternative, and improved, adjuvants.

Surprisingly the inventors have found that selected molecular adjuvants are able to enhance the immunogenicity and efficacy of the immune response induced by vectored vaccines when encoded by a vector. Suitable molecular adjuvants include co-stimulatory molecules, cytokines and molecules from the toll-like receptor signalling pathways. Suitable vectors include naked DNA, such as plasmid DNA, modified vaccinia Ankara (MVA) virus, and adenovirus. The combination of a vectored vaccine encoding (i) an appropriate antigen and (ii) a selected molecular adjuvant, results in a vaccine/immunogenic composition capable of inducing an improved antigen-specific immune response, in particular an improved CD8 T cell response, and/or improved disease protection, when administered to an organism.

Reference herein to a molecular adjuvant refers to an adjuvant expressed from a gene which forms part of the vector. In all aspects of the invention the adjuvant used is a molecular adjuvant, that is, the adjuvant is encoded by a gene on the vector.

### IL-7 or IL-15 as Molecular Adjuvants

According to a first aspect, the present invention provides an immunogenic composition comprising one or more vectors encoding IL-7 and/or IL-15 and one/or more target antigens. In one embodiment, the immunogenic composition comprises a vector encoding IL-7 and/or IL-15 and a target antigen.

According to a further aspect, the invention provides a vaccine composition comprising one or more vectors encoding IL-7 and/or IL-15 and a further vector encoding one or more target antigens. In one embodiment, the invention provides a vaccine composition comprising a vector encoding IL-7 and/or IL-15 and a target antigen.

According to a yet further aspect, the invention provides a composition comprising one or more vectors encoding one or more target antigens and IL7 and/or IL15 for use in inducing or amplifying an immune response to one or more target antigens in a mammal. In one embodiment, the invention provides a composition comprising a vector encoding IL-7 and/or IL-15 and a target antigen for use in inducing or amplifying an immune response to one or more target antigens in a mammal.

According to another aspect, the invention provides a method of inducing an immune response in an organism, such as a mammal, comprising the step of administering a composition according to one of the preceding three aspects of the invention to the mammal.

According to a further aspect, the invention provides the use of one or more vectors encoding one or more target antigens and IL-7 and/or IL-15 in the preparation of a medicament for use in inducing or amplifying an immune response in an organism, such as a mammal. In one embodiment one vector is used which encodes IL-7 and/or IL-15 and one or more target antigens.

According to another aspect, the invention provides a method of inducing an immune response in an organism, such as a mammal, comprising the steps of exposing the organism to a priming composition that comprises one or more vectors encoding one or more antigens, and then boosting the immune response by administering a boosting composition comprising one or more vectors encoding the same antigen or antigens as the priming composition, wherein either the vector used in the priming composition or the vector used in the boosting composition, or both vectors, also encode IL-7 and/or IL-15. Alternatively, the priming or boosting composition may comprise a further vector encoding IL-7 and/or IL-15.

Preferably, the type of vector used in the priming composition is different to the type of vector used in the boosting composition but the target antigen or epitope in both is the same (heterologous prime boost). Preferably the vectors are derived from different sources, for example, from different types of virus. For example, one vector may be an MVA vector and the other vector may be an adenovirus or DNA vector, or different strains of adenovirus may be used to prime and to boost. Preferably, the vector used in the prime is an adenovirus, and the vector used in the boost is MVA. The use of different vector types may enhance the immunogenicity provided by the prime-boost immunisation regime in animals and humans (McConkey et al. Nature Medicine 2003). In another embodiment, the same vector or vectors may be used in the prime and boost immunisations, the regime may then be referred to as an homologous prime boost.

Preferably the one or more antigens and the IL-7 and/or IL-15 are operably linked to a promoter in the vector.

In the compositions of this aspect of the invention a gene encoding IL-7 or IL-15 is included in the vector as a molecular adjuvant. The vector is arranged such that expression of the gene encoding IL-7 or IL-15 produces the IL-7 or IL-15 protein in an organism to which the composition is administered, the expressed adjuvant protein may then act to enhance the immune response elicited in the organism by the target antigen encoded by the same or a different vector.

Preferably the (host) organism is a mammal, this may be a human or non-human mammal or a bird such as a chicken. A non-human mammal may include a horse, cow, sheep, pig, goat, mouse, rat, monkey or chimpanzee.

In addition to their potential use as vaccines, immunogenic compositions according to the invention may be useful a) as diagnostic reagents; b) in adoptive T cell therapy protocols; and c) as a measure of immune competence of the vaccinee.

IL-7 and IL-15 share a common receptor component, the common gamma chain, and both are well known to impact on T cell memory in mammals (McKinlay et al. Immunology. 2007 Mar;120(3):392-403.) and thus both would be expected to have similar effects on adjuvanting T cell responses.

The immune response induced or amplified in an organism may be a cellular immune response and/or a humoral immune response. If a cellular immune response is induced or amplified, the composition may, when administered to an organism, induce a T cell response against an antigen encoded by the vector in the composition. Preferably the T cell response is a CD8 + and/or a CD4 + T cell response. The CD4 + response may be a gamma interferon response. The CD8 response may be induced or amplified by using an antigen that contains a CD8 epitope. Preferably the induced T cells are polyfunctional and express multiple cytokines such as interferon-gamma, TNF-alpha and interleukin-2. Preferably the immune response is protective, that is, it serves to protect, either reduce or prevent, the organism from developing an infection or disease related to the antigen encoded by a vector in the composition.

If a humoral response is amplified or induced by a composition of the invention, preferably it is a TH1 biased antibody response to a target antigen.

The immune response may be directed to a pathogen or a cancer. The pathogen may be infectious.

The immune response may be assessed by determining antigen-specific IFNγ secretion levels by lymphocytes, or by assaying for other cytokines secreted/induced in an antigen-specific manner. Other cytokines which may be secreted/induced in an antigen-specific manner include IL-2, IL-4, IL-12, and TNF-alpha. The aforementioned methods are just some examples of how induction of the cellular immune system may be monitored, and are not intended to be exhaustive.

It has been surprisingly found that a composition according to the invention, comprising a vector encoding the molecular adjuvant IL-7 and an antigen results in a dramatic improvement in the immunogenicity of the antigen.

The one or more vectors may be a DNA vector or may be a non-replicating or a replication impaired viral or bacterial vector, or may be a combination of vectors. The terms “non-replicating” or “replication impaired” as used herein mean that the vector is not capable of replication to any significant extent in a host organism, and in particular is unable to cause serious infection in the host. The host organism is preferably a human, wherein the terms “non-replicating” or “replication impaired” mean that the vector is not capable of replication to any significant extent in normal human cells.

Replication of a virus, and thus a viral vector, can be measured in two ways: (i) DNA synthesis, and (ii) viral titre. For adenovirus a non-replicating or a replication impaired viral vector, may exhibit a significant reduction in viral titre on infection of cells, such as HeLa cells, which are not permissive for the replication of the replication-deficient adenovirus. For poxvirus, a non-replicating or a replication impaired viral vector, may exhibit a 2 log reduction in viral titre in HELA cells (a human cell line) compared to the Copenhagen strain of the vaccinia virus. Examples of poxviruses which fall within this definition are MVA, NYVAC and avipox.

The one or more vectors encoding the IL-7 and/or IL-15 and the one or more target antigens may be the same type of vector, for example, they may all be adenoviral vectors. This would simplify administration. Alternatively the one or more vectors may comprise two or more different types of vector, for example a DNA and a viral vector, or two different types of viral vector or two different strains of adenovirus. Where more than one vector is used, each antigen is provided in a single vector type only.

Preferably one or more of the vectors is a viral vector, preferably one or more of the vectors is a non-replicating or a replication impaired viral vector

Preferably the viral vector is based on a virus selected from the group comprising adenoviruses; vaccinia derived viruses, such as, MVA or NYVAC; avipox viruses, such as, canary pox or fowl pox; alpha viruses; herpes viruses; flaviviruses; retroviruses and influenza viruses. Adenoviral vectors may include non-replication or replication impaired human or simian adenoviruses.

Preferably if one or more of the vectors is a viral vector, at least one vector is an adenovirus or an MVA virus.

In addition to viral vectors, the one or more vectors may be bacterial or DNA vectors. A bacterial vector may comprise recombinant Salmonella, recombinant Shigella or recombinant BCG. A DNA vector may comprise plasmid DNA.

Viruses or bacteria that are non-replicating or replication impaired may have arisen naturally or may have been produced artificially, for example, by genetic manipulation.

Preferably the adjuvant is IL-15 and is encoded by an adenovirus or an MVA vector. More preferably the vector is an adenovirus. The vector may also encode one or more target antigens and/or IL7. Alternatively, the one or more target antigens and/or IL7 may be encoded on a separate vector.

Wherein the one or more antigens and IL7 and/or IL15 are encoded on two or more vectors, the vectors may be administered simultaneously or sequentially. One vector may be encode IL7 and/or IL15 and another vector may encode one or more target antigens. For example, in one embodiment an antigen may be encoded on a first vector, such as an adenoviral vector, and an adjuvant, such as a IL7 or IL15, may be encoded on a second vector, which may also be an adenoviral vector. Alternatively, the first and second vectors may be different vector types, for example one may be an adenovirus and the other may be MVA. The first and second vectors may be administered to an organism simultaneously or sequentially, wherein sequential administration requires the two vectors to be administered within 3 days of each other, preferably within 2 days, more preferably within 24 hours. Simultaneous administration is intended to mean administration within 2 hours or at the same time. The two or more vectors may be administered at the same or different sites, and may be administered by the same or a different route.

Where two or more vectors are used as a mixture, the two vectors may be of the same or different types. Where two or more vectors are used, each antigen is encoded by only one type of vector.

Preferably the one or more antigens encoded by the vector is derived from a pathogen, such as a virus, a bacterium or a fungus, or from a disease, such as cancer. For example, one or more of the antigens encoded by the vector may be a protein or polypeptide or epitope derived from one or more of the following pathogens, HIV type 1 and 2 (HIV-1 and HIV-2 respectively), Human T Cell Leukaemia Virus types 1 and 2 (HTLV-1 and HTLV-2 respectively), Herpes Simplex Virus types 1 and 2 (HSV-1 and HSV-2 respectively), a picornavirus, a hepadnavirus, a flavivirus, Haemophilus influenzae, human papilloma virus, Lassa fever virus, HBV, HCV, EBV, CMV, foot and mouth disease virus, Epstein Barr virus, Treponema pallidum, Neisseria gonorrhoea, a Plasmodium, a pathogenic Steptococcus species, M. Tuberculosis, Chlamydia trachomatis, Toxoplasma gondii, Leishmania species, Cytomegalovirus and Candida albicans. Alternatively, or additionally, the antigen may be a cancer antigen or epitope, a measles, mumps and/or rubella antigen or epitope, a tetanus antigen or epitope, a diphtheria antigen or epitope, an Ebola antigen or epittope, a hepatitis A, B or C antigen or epitope, a polio antigen or epitope and/or an antigen or epitope relating to any other disease.

The one or more antigens or epitopes may be derived from bacteria selected from the list comprising mycobacteria, pneumococci, meningococci, Burkholderia, shigella and salmonella.

The one or more antigens or epitopes may be derived from an organism that causes a parasitic disease, this includes plasmodia, theileria, schistosomes, leishmania, eimeria

A cancer antigen or epitope may include or be part of a human heat shock protein or a tumour associated antigen, such as, CEA, PSA, Muc 1 or Her2neu. The cancer antigen or epitope may be derived from one of the following cancers, lung, breast, kidney, colon, rectum, bone, brain, thyroid and haematological malignancies such as leukaemias and lymphomas

The antigen may be naturally expressed by the vector. For example, if the vector is an adenovirus, the antigen may be an adenovirus protein which may confer immunity against subsequent infection and/or disease caused by an adenovirus of the same or similar strain. Alternatively, or additionally, the antigen may be exogenous to the vector.

The vector may encode one or more antigens. If the vector encodes more than one antigen, the antigens may be derived from the same pathogen or disease, or from different pathogens or diseases.

The method of the invention may be used to immunise against diseases in which T cell responses play a protective role. Such diseases include, but are not limited to, malaria, infection and/or disease caused by the viruses HIV, herpes simplex, herpes zoster, hepatitis C, hepatitis B, influenza, Epstein Barr virus, measles, dengue, and HTLV-1; infection and/or disease caused by the bacterium Mycobacterium tuberculosis and Listeria spp; infection and/or disease caused by encapsulated bacteria such as streptococcus and haemophilus; infection and/or disease caused by parasites such as Leishmania, Toxoplasma and Trypanosoma. The method of the invention may also be used to immunise against certain forms of cancer, for example, melanoma, lymphomas and leukaemias, cancers of the lung, breast and colon, or against other diseases.

Preferably the immunogenic or vaccine composition is for use in therapeutic or prophylactic treatments or both.

The immune response elicited by any method of the invention may be therapeutic or prophylactic or both.

An immunogenic or vaccine composition according to the invention may be for oral, systemic, parenteral, topical, mucosal, intramuscular, intravenous, intraperitoneal, intradermal, subcutaneous, intranasal, intravaginal, intrarectal, transdermal, sublingual, inhalation or aerosol administration.

A composition according to the invention may be administered to a subject/organism in the form of a pharmaceutical composition. In addition to the immunogenic or vaccine composition, a pharmaceutical composition preferably comprises one or more physiologically and/or pharmaceutically effective carriers, diluents, excipients or auxiliaries which facilitate processing and/or delivery of the antigen and/or adjuvant.

Determination of an effective amount of an immunogenic or vaccine composition for administration to an organism is well within the capabilities of those skilled in the art. For example, for mouse to humans, a DNA vaccination dose may comprise from about 0.1 μg to about 10 mg. For an adenoviral vector the vaccination dose may be between about 1×10⁶ and 1×10¹⁶ viral particles per animal. For an MVA vector the vaccination dose may be between about 1×10² and 1×10¹⁰ pfu per animal.

In addition, to the IL-7 and/or IL-15 molecular adjuvants, a further molecular adjuvant may be included on the same or a further vector. The further molecular adjuvant may be a co-stimulatory molecule or a TLR signalling pathway component.

A composition according to the invention may be administered with a non-vectored adjuvant.

A composition according to the invention may be used in isolation, or it may be combined with one or more other immunogenic or vaccine compositions, and/or with one or more other therapeutic regimes.

According to a further aspect the invention provides a kit for use in inducing an immune response in an organism, comprising an immunogenic or vaccine composition according to the invention and instructions relating to administration.

According to a yet further aspect, the invention provides a pharmaceutical composition comprising an immunogenic or a vaccine composition according to the invention and one or more physiologically effective carriers, diluents, excipients or auxiliaries.

According to another aspect, the invention provides the use of an immunogenic composition according to the invention in the preparation of a medicament for the treatment and/or prevention of infection and/or disease related to the antigen encoded by the vector in the immunogenic composition.

Where the antigen encoded by the vector in the composition is from a pathogen, the medicament may be intended/used to confer protection from infection and/or from disease caused by the pathogen from which the antigen of interest is derived. Alternatively, where the antigen encoded by the vector in the composition is a cancer antigen or an antigen associated with a particular disease, the medicament may be intended/used to confer protection from, and/or to treat, the cancer or the particular disease from which the antigen is derived.

The medicament or composition may be a vaccine.

According to another aspect the invention provides the use of an immunogenic composition according to the invention in the treatment and/or prevention of infection or disease related to the antigen encoded by a vector in the immunogenic composition.

Preferably, in a use according to the invention the composition or medicament induces an immune response when administered to an organism.

In an alternative embodiment the invention provides an immunogenic or a vaccine composition comprising one or more vectors, as defined herein, encoding two or more molecular adjuvants and one or more target antigen, wherein at least one of the molecular adjuvants is IL-7 or IL-15 and wherein one or more of the further adjuvants are selected from the group comprising other cytokines, co-stimulatory molecules and TLR signalling pathway components.

Preferably co-stimulatory molecules and TLR signalling pathway components are as described herein.

The two or more molecular adjuvants may be from the same group, for example, they may all be cytokines, or they may be a mixture of these molecules, for example, a cytokine and a co-stimulatory molecule and/or a TLR signalling pathway component.

The vector may comprise a co-stimulatory molecule and a TLR signalling pathway component.

The vector may comprise a cytokine and a co-stimulatory molecule.

The vector may comprise a cytokine and a TLR signalling pathway component.

In a still further embodiment, the two or more molecular adjuvants may be encoded on separate vectors, or on the same vector. The two or more molecular adjuvants may be on the same or different vectors to the one or more target antigens.

### Co-Stimulatory Receptor Ligands as Molecular Adjuvants

According to a further aspect, the invention provides a method of inducing or amplifying an immune response to a target antigen in a mammal, comprising administering to the mammal a first vector encoding one or more target antigens and a second vector encoding one or more co-stimulatory molecules.

According to a yet further aspect, the invention provides a composition comprising two or more vectors, wherein a first vector encodes one or more target antigens and a second vector encodes one or more co-stimulatory molecules, for use in inducing or amplifying an immune response to one or more target antigens in a mammal.

According to another aspect, the invention provides the use of a first vector encoding one or more target antigens and a second vector encoding one or more co-stimulatory molecules in the preparation of a medicament for use in inducing or amplifying an immune response in a mammal.

According to a further aspect, the invention provides an immunogenic composition comprising a first vector encoding one or more target antigens and a second vector encoding one or more co-stimulatory molecules.

The first and second vector may be selected from the group comprising a DNA vector, a pox viral vector, a vaccinia derived viral vector and an adenoviral vector. Preferably the first and second vectors are either adenoviral or MVA vectors. Preferably the first and second vectors are not fowl pox virus vectors.

The first and second vectors may be co-administered at substantially the same time, preferably within 2 hours of each other. The first and second vectors may be co-administered in a single administration.

The first and second vectors may be administered at different times. Preferably within 72 hours of each other.

The first and second vectors may be administered at different sites and/or by different routes.

The first and second vectors may be the same type of vector, or they may be different types of vector.

According to a further aspect, the present invention provides an immunogenic composition comprising a non-replicating or replication impaired viral vector encoding one or more co-stimulatory molecule and one or more target antigens.

According to a further aspect, the invention provides a vaccine composition comprising a non-replicating or replication impaired viral vector encoding one or more co-stimulatory molecule and one or more target antigens.

According to a yet further aspect, the invention provides a composition comprising a non-replicating or replication impaired viral vector encoding one or more co-stimulatory molecule and one or more target antigens for use in inducing or amplifying an immune response to one or more of the target antigens.

According to another aspect, the invention provides a method of inducing an immune response in an organism, such as a mammal, comprising the step of administering a composition according to the invention to the organism.

According to yet another aspect, the invention provides the use of a non-replicating or replication impaired viral vector encoding one or more co-stimulatory molecule and one or more target antigens in the preparation of a medicament for use in inducing or amplifying an immune response to one or more of the target antigens.

Preferably a non-replicating or replication impaired viral vector comprises an adenoviral vector or replication deficient orthopox viral vector, such as MVA.

According to another aspect, the invention provides a method of inducing an immune response in an organism, such as a mammal, comprising the steps of exposing the organism to a priming composition that comprises a non-replicating or replication impaired viral vector encoding an antigen, and then boosting the immune response by administering a boosting composition comprising a non-replicating or replication impaired viral vector encoding the same antigen as the priming composition, wherein either the vector used in the priming composition or the vector used in the boosting composition, or both vectors, also encodes one or more co-stimulatory molecule. Alternatively, the priming or boosing composition may comprise a further vector encoding one or more co-stimulatory molecules.

Preferably, the type of viral vector used in the priming composition is different to the type of viral vector used in the boosting composition, but the target antigen or epitope in both is the same (heterologous prime boost. Preferably the viral vector used in the priming composition is derived from a different virus to the virus used in the boosting composition. The use of different vectors may enhance the immunogenicitiy provided by the prime-boost regime in animals and humans (McConkey et al. Nature Medicine 2003)

However, in some embodiments, the viral vectors used in the prime and boost immunisation may be derived from the same virus. This may simplify administration. In another embodiment, each of the prime and boost immunisations may comprise at least two vectors, wherein the first vector encodes one or more target antigens, and the second vector encodes one or more co-stimulatory molecules. In each administration the first and second vectors may be the same or different.

A co-stimulatory molecule may be a cell surface receptor of a T or B lymphocytes which is engaged in immunosynapsis with antigen presenting cells (APCs) and is involved in modulating the initial response of these cells to antigen by stimulating T-cell proliferation and differentiation. Well recognised receptors which provide co-stimulatory activity include CD28, CTLA-4, CD27, OX-40, 4-1BB, CD30, GITR and NKG2D. Signalling of these receptors is initiated following engagement of the receptors by their cognate ligands, respectively B7-1 and B7-2, CD70, OX40L, 4-1BBL, CD30L, GITRL and Raet1e. Accordingly, a co-stimulatory molecule according to the invention may be selected from the group comprising B7-1, B7-2, CD70, OX40L, 4-1BBL, CD30L, GITRL, Raet1e, hULBP, MICA, MICB, BAFF and TLA. Wherein Raet1e, MICA, MICB and hULBP are NKG2D ligands.

A co-stimulatory molecule may be a B7 superfamily member or a TNFR superfamily member.

A co-stimulatory molecule may also be a molecule which is a member of the ephrin family, such as one of the Ephrin A or Ephrin B molecules, or the molecule variously described as Nec12, Tsc1c1, SynCaM, SgIGSF or IGSF4, or another member of the IGSF4 family.

Preferably the vector encodes one, two, three, four, five, six or more different co-stimulatory molecules, and one or more antigen. Preferably the one or more co-stimulatory molecules are selected from the group comprising B7-1, B7-2, CD70, OX40L, 4-1BBL, CD30L, GITRL, Raet1e, hULBP, MICA, MICB, BAFF and TLA.

Preferably, the co-stimulatory molecules are selected from the group comprising 4-1BBL, OX40L, CD70 or CD30.

Preferably the co-stimulatory molecules comprise NKG2D ligands, preferably Raet1e and hULBP.

In the compositions of the invention co-stimulatory molecules are included as molecular adjuvants, which serve to enhance the immune response elicited by an antigen in the composition.

Preferably the organism is a mammal, this may be a human or non-human mammal or a bird such as a chicken. A non-human mammal may include a horse, cow, sheep, pig, goat, mouse, rat, monkey or chimpanzee.

In addition to their potential use as vaccines, immunogenic compositions according to the invention may be useful a) as diagnostic reagents; b) in adoptive

T cell therapy protocols; and c) as a measure of immune competence of the vaccinee.

The immune response induced or amplified in an organism may be a cellular immune response and/or a humoral immune response. If a cellular immune response is induced or amplified, the composition may, when administered to an organism, induce a T cell response against an antigen encoded by the vector in the composition. Preferably the T cell response is a CD8 + and/or a CD4 + T cell response. Preferably the induced T cell response is polyfunctional in that induced cells produce interferon-gamma, TNF-alpha and interleukin-2. Preferably the immune response is protective, that is, it serves to protect, either reduce or prevent, the organism from developing an infection or disease related to the antigen encoded by the vector in the composition

If a humoral response is amplified or induced by a composition of the invention, preferably it is a TH1 biased antibody response to a target antigen.

The immune response may be directed to a pathogen or a cancer. The pathogen may be infectious.

The immune response may be assessed by determining antigen-specific IFNγ secretion levels by lymphocytes, or by assaying for other cytokines secreted/induced in an antigen-specific manner. Other cytokines which may be secreted/induced in an antigen-specific manner include IL-2, IL-4, IL-12, and TNF-alpha. The aforementioned methods are just some examples of how induction of the cellular immune system may be monitored, and are not intended to be exhaustive.

The terms “non-replicating” or “replication impaired” as used herein mean that the viral vector is not capable of replication to any significant extent in a host organism, and in particular is unable to cause serious infection in the host. The host organism is preferably a human, wherein the terms “non-replicating” or “replication impaired” mean that the vector is not capable of replication to any significant extent in normal human cells.

Replication of a virus, and thus a viral vector, can be measured in two ways: (i) DNA synthesis, and (ii) viral titre. For adenovirus a non-replicating or a replication impaired viral vector, may exhibit a significant reduction in viral titre on infection of cells, such as HeLa cells, which are not permissive for the replication of the replication-deficient adenovirus. For poxvirus, a non-replicating or a replication impaired viral vector, may exhibit a 2 log reduction in viral titre in HELA cells (a human cell line) compared to the Copenhagen strain of the vaccinia virus. Examples of poxviruses which fall within this definition are MVA, NYVAC and avipox.

For first or second vector may be a viral vector. Preferably the viral vector is based on a virus selected from the group comprising adenoviruses; vaccinia derived viruses, such as, MVA or NYVAC; avipox viruses, such as, canary pox or fowl pox; alpha viruses; herpes viruses; flaviviruses; retroviruses and influenza viruses. Adenoviral vectors may include non-replication or replication impaired human or simian adenoviruses.

Preferably the viral vector is an adenovirus or an orthopox virus such as the MVA or an avipoxvirus vector. Preferably the viral vector is not the fowl pox virus.

Viruses that are non-replicating or replication impaired may have arisen naturally or they may have been produced artificially, for example, by genetic manipulation.

In addition to viral vectors, the one or more vectors may be bacterial or DNA vectors. A bacterial vector may comprise recombinant Salmonella, recombinant Shigella or recombinant BCG. A DNA vector may comprise plasmid DNA.

Preferably the one or more antigens encoded by the vector is derived from a pathogen, such as a virus, a bacterium or a fungus, or from a disease, such as cancer. For example, one or more of the antigens encoded by the vector may be a protein or polypeptide or epitope derived from one or more of the following pathogens, HIV type 1 and 2 (HIV-1 and HIV-2 respectively), Human T Cell Leukaemia Virus types 1 and 2 (HTLV-1 and HTLV-2 respectively), Herpes Simplex Virus types 1 and 2 (HSV-1 and HSV-2 respectively), a picornavirus, a hepadnavirus, a flavivirus, Haemophilus influenzae, human papilloma virus, Lassa fever virus, HBV, HCV, EBV, CMV, foot and mouth disease virus, Epstein Barr virus, Treponema pallidum, Neisseria gonorrhoea, a Plasmodium, a pathogenic Steptococcus species, M. Tuberculosis, Chlamydia trachomatis, Toxoplasma gondii, Leishmania species, Cytomegalovirus and Candida albicans. Alternatively, or additionally, the antigen may be a cancer antigen or epitope, a measles, mumps and/or rubella antigen or epitope, a tetanus antigen or epitope, a diphtheria antigen or epitope, an Ebola antigen or epittope, a hepatitis A, B or C antigen or epitope, a polio antigen or epitope and/or an antigen or epitope relating to any other disease.

The one or more antigens or epitopes may be derived from bacteria selected from the list comprising mycobacteria, pneumococci, meningococci, Burkholderia, shigella and salmonella.

The one or more antigens or epitopes may be derived from an organism that causes a parasitic disease, this includes plasmodia, theileria, schistosomes, leishmania, eimeria

A cancer antigen epitope may include or be part of a human heat shock protein or a tumour associated antigen, such as, CEA, PSA, Muc 1 or Her2neu. The cancer antigen or epitope may be derived from one of the following cancers, lung, breast, kidney, colon, rectum, bone, brain, thyroid and haematological malignancies such as leukaemias and lymphomas.

The antigen may be naturally expressed by the vector. For example, if the vector is an adenovirus, the antigen may be an adenovirus protein which may confer immunity against subsequent infection and/or disease caused by an adenovirus of the same or similar strain. Alternatively, or additionally, the antigen may be exogenous to the vector.

The vector may encode one or more antigens. If the vector encodes more than one antigen, the antigens may be derived from the same pathogen or disease, or from different pathogens or diseases.

The method of the invention may be used to immunise against diseases in which T cell responses play a protective role. Such diseases include, but are not limited to, malaria, infection and/or disease caused by the viruses HIV, herpes simplex, herpes zoster, hepatitis C, hepatitis B, influenza, Epstein Barr virus, measles, dengue, and HTLV-1; infection and/or disease caused by the bacterium Mycobacterium tuberculosis and Listeria spp; infection and/or disease caused by encapsulated bacteria such as streptococcus and haemophilus; infection and/or disease caused by parasites such as Leishmania, Toxoplasma and Trypanosoma. The method of the invention may also be used to immunise against certain forms of cancer, for example, melanoma, lymphomas and leukaemias, cancers of the lung, breast and colon, or against other diseases.

Preferably the immunogenic or vaccine composition is for use in therapeutic or prophylactic treatments or both.

The immune response elicited by any method of the invention may be therapeutic or prophylactic or both.

An immunogenic or vaccine composition according to the invention may be for oral, systemic, parenteral, topical, mucosal, intravenous, intramuscular, intraperitoneal, intradermal, subcutaneous, intranasal, intravaginal, intrarectal, transdermal, sublingual, or inhalation administration.

A composition according to the invention may be administered to a subject/organism in the form of a pharmaceutical composition. In addition to the immunogenic or vaccine composition, a pharmaceutical composition preferably comprises one or more physiologically and/or pharmaceutically effective carriers, diluents, excipients or auxiliaries which facilitate processing and/or delivery of the antigen and/or adjuvant.

Determination of an effective amount of an immunogenic or vaccine composition for administration to an organism is well within the capabilities of those skilled in the art. For example, for mouse to humans, a DNA vaccination dose may comprise from about 0.1 μg to about 10 mg. For an adenoviral vector the vaccination dose may be between about 1×10⁶ and 1×10¹⁶ viral particles per animal. For an MVA vector the vaccination dose may be between about 1×10² and 1×10¹⁰ pfu per animal.

A composition according to the invention may be used in isolation, or it may be combined with one or more other immunogenic or vaccine compositions, and/or with one or more other therapeutic regimes.

A composition may further encode an additional molecular adjuvant, this may be on the same vector to a co-stimulatory molecule and/or a target antigen, or on another vector. The additional molecular adjuvant may be a cytokine such as IL-7 or IL-15, or a TLR signalling pathway component as described herein.

A composition according to the invention may be administered with a non-vectored adjuvant.

In an alternative embodiment the immunogenic or vaccine composition according to the invention may comprise a first vector encoding one or more co-stimulatory molecule and a second vector encoding one or more antigens, wherein the first or second vector is a non-replicating or replication impaired viral vector. Preferably both vectors are a non-replicating or replication impaired viral vector. If both vectors are viral vectors they may be derived from the same or different viruses. Preferably the viruses are selected from an adenovirus and a replication-impaired (ie non-replicating) orthopox virus.

According to a further aspect the invention provides a kit for use in inducing an immune response in an organism, comprising an immunogenic or vaccine composition according to the invention and instructions relating to administration.

According to a yet further aspect, the invention provides a pharmaceutical composition comprising an immunogenic or a vaccine composition according to the invention and one or more physiologically effective carriers, diluents, excipients or auxiliaries.

According to another aspect, the invention provides the use of an immunogenic composition according to the invention in the preparation of a medicament for the treatment and/or prevention of infection and/or disease related to the antigen encoded by the vector in the immunogenic composition.

Where the antigen encoded by the vector in the composition is from a pathogen, the medicament may be intended/used to confer protection from infection and/or from disease caused by the pathogen from which the antigen of interest is derived. Alternatively, where the antigen encoded by the vector in the composition is a cancer antigen or an antigen associated with a particular disease, the medicament may be intended/used to confer protection from, and/or to treat, the cancer or the particular disease from which the antigen is derived.

The medicament or composition may be a vaccine.

According to another aspect the invention provides the use of an immunogenic composition according to the invention in the treatment and/or prevention of infection or disease related to the antigen encoded by a vector in the immunogenic composition.

Preferably, in a use according to the invention the composition or medicament induces an immune response when administered to an organism.

In an alternative embodiment of the invention the gene encoding the molecular adjuvant and the gene encoding the antigen may be provided on different vectors. Preferably, at least one of the vectors is an adenovirus vector or an MVA vector.

### Toll-Like Receptor (TLR) Signalling Pathway Components as Molecular Adjuvants

According to another aspect, the invention provides one or more vectors encoding one or more TLR signalling pathway components and one or more target antigens, wherein at least one target antigen includes a CD8 T cell epitope.

According to a further aspect, the present invention provides an immunogenic composition comprising one or more vectors encoding one or more TLR signalling pathway components and one or more target antigens. Preferably, at least one target antigen includes a CD8 T cell epitope. In one embodiment, the present invention provides an immunogenic composition comprising a vector encoding a TLR signalling pathway components and a target antigen. Preferably, at least one target antigen includes a CD8 T cell epitope.

According to a further aspect, the invention provides a vaccine composition comprising an immunogenic composition according to the preceding aspect, for example, comprising a vector encoding a TLR signalling pathway component and a target antigen.

Preferably the composition of any of the preceding three aspects of the invention may be used to induce or amplify a CD8 T cell response to a target antigen.

According to another aspect, the invention provides a method of inducing or amplifying an immune response, preferably a CD8 T cell response, in an organism, preferably a mammal, comprising the step of administering an immunogenic or vaccine composition according to the invention to the organism.

According to a further aspect the invention provides the use of one or more vectors encoding one or more TLR signalling pathway component and one or more target antigens, wherein at least one target antigen includes a T cell epitope, in the preparation of a medicament for use in inducing or amplifying a CD8 T cell response to a target antigen.

According to another aspect, the invention provides a method of inducing an immune response in an organism, preferably a mammal, comprising the steps of exposing the organism to a priming composition that comprises one or more vectors encoding one or more antigens, and then boosting the immune response by administering a boosting composition comprising one or more vectors encoding the same antigens as the priming composition, wherein either the one or more vectors used in the priming composition or the one or more vectors used in the boosting composition, or both, also encode one or more TLR signalling pathway components. Alternatively, the priming and boosting composition may comprise a further vector encoding one or more TLR signalling pathway components.

Preferably, the type of vector used in the priming composition is different to the type of vector used in the boosting composition but the target antigen in both is the same (heterologous prime boost regimen). Preferably the vectors are derived from different sources, for example, from different types of virus. For example, one vector may be an MVA vector and the other vector may be an adenovirus or DNA vector. The use of different vector types may enhance the immunogenicity provided by the prime-boost immunisation regime in animals and humans (McConkey et al. Nature Medicine 2003). If the same vector is used in both the prime and boost immunisations, it is referred to as homologous.

Preferably the one or more antigens and the one or more TLR signalling pathway components are operably linked to promoters in the one or more vectors.

However, in some embodiments, the two vectors may be the same or derived from the same source, for example, both may be adenovirus vectors. This may simplify administration.

In the compositions of the invention the TLR signalling pathway component is included as a molecular adjuvant. That is, the expression of the TLR signalling pathway component in the organism to which the composition is administered serves to enhance the immune response elicited by the antigen in the composition.

Toll-like receptors (TLRs) are well-characterised receptors recognizing components of microbes (Banerjee A and Gerondakis S Immunology Cell Biol. 2007). A TLR signalling pathway component according to the invention may include one or more of NIK, TIRAP, TRAF6, TRAF3, TRIF, IRAK(s), TAB1, TAB2, TAB3, TBK1, IKKi, TIP1, NEMO, IRF(s), UbC13, Uev1A, Pellino-1, Pellino-2, TIRP, SARM, Myd88, TRAM, Act-1/CIKS, TAK1, MAP3K10 and MAP3K11. Preferably the TLR signalling pathway component is not Myd88.

A vector according to the invention may encode two or more TLR signalling pathway components. Preferably when two or more TLR signalling pathway components are encoded by one or more vectors, the one or more vectors are a DNA or adenovirus vectors.

The vector may encode one or more of TRAM, TRAF6, TIRAP, and TAK1 and one or more additional molecule that enhances antigen immunogenicity. The one or more additional molecule that enhances antigen immunogenicity may be a further TLR signalling pathway component, or it may by a different kind of molecular adjuvant, such as a cytokine or a co-stimulatory molecule.

A vector may encode the TRAM and TRAF6 molecules or the TRAM and TAK1 molecules. Additionally, a composition may encode the TRAM and TRAF6 molecules, or the TRAM and TAK1 molecules, on different vectors.

The one or more molecules that enhance the antigen immunogenicity may be encoded on the same vector as the TLR signalling pathway component or on a different vector. For example, a first molecule that enhances antigen immunogenicity may be encoded on a first vector and a second molecule that enhances antigen immunogenicity may be encoded on a second vector. The first and second vector may be the same type of vector, for example both viral vectors, or both DNA vectors, or they may be different vector types, for example one vector may be a DNA vector and the other may be an adenovirus vector. The first molecule and the second molecule may both be TLR signalling pathway components, or one may be a different kind of molecular adjuvant, such as a cytokine or a co-stimulatory molecule.

Preferably the organism is a human or non-human mammal or a bird such as chicken. A non-human mammal may include a horse, cow, sheep, pig, goat, mouse, rat, monkey or chimpanzee.

In addition to their potential use as vaccines, immunogenic compositions according to the invention may be useful a) as diagnostic reagents; b) in adoptive T cell therapy protocols; and c) as a measure of immune competence of the vaccinee.

The immune response induced or amplified in an organism, such as a mammal, may be a cellular immune response and/or a humoral immune response. If a cellular immune response is induced or amplified, the composition may, when administered to an organism, induce a T cell response against an antigen encoded by the vector in the composition. Preferably the T cell response is a CD8 + and/or a CD4 + T cell response. The CD4 response may be a CD4 gamma interferon response. Preferably the T-cell response is polyfunctional, in that the induced cells produce multiple cytokines, such TNF-alpha and interleukin-2 in addition to interferon-gamma. Preferably if a CD8 response is induced or amplified, one or more of the antigens encode a T cell epitope. Preferably the immune response is protective, that is, it serves to protect, either reduce the likelihood of or prevent, the organism from developing an infection or disease related to the antigen encoded by the vector in the composition.

If a humoral response is induced or amplified, preferably it is a TH1 biased antibody response directed to one or more of the target antigens.

The immune response may be directed to a cancer or a pathogen. The pathogen may be infectious.

The immune response may be assessed by determining antigen-specific IFNγ secretion levels by lymphocytes, or by assaying for other cytokines secreted/induced in an antigen-specific manner. Other cytokines which may be secreted/induced in an antigen-specific manner include IL-2, IL-4, IL-12, and TNF-alpha. The aforementioned methods are just some examples of how induction of the cellular immune system may be monitored, and are not intended to be exhaustive.

It has been surprisingly found that a composition according to the invention, comprising a vector encoding the molecular adjuvant the TLR signalling pathway component and an antigen results in a dramatic improvement in immunogenicity of the antigen.

Replication of a virus, and thus a viral vector, can be measured in two ways: (i) DNA synthesis, and (ii) viral titre. For adenovirus a non-replicating or a replication impaired viral vector may exhibit a significant reduction in viral titre on infection of cells, such as HeLa cells, which are not permissive for the replication of the replication-deficient adenovirus. For poxvirus a non-replicating or a replication impaired viral vector may exhibit a 2 log reduction in viral titre in HELA cells (a human cell line) compared to the Copenhagen strain of the vaccinia virus. Examples of poxviruses which fall within this definition are MVA, NYVAC and avipox.

Preferably one or more of the vectors is a viral vector, preferably one or more of the vectors is a non-replicating or a replication impaired viral vector.

Preferably the viral vector is based on a virus selected from the group comprising adenoviruses; vaccinia derived viruses, such as, MVA or NYVAC; avipox viruses, such as, canary pox or fowl pox; alpha viruses; herpes viruses; flaviviruses; retroviruses and influenza viruses. Adenoviral vectors may include non-replication or replication impaired human or simian adenoviruses.

Preferably if the vector is a viral vector it is an adenovirus or an MVA virus.

Alternative, one or more of the vectors may be a bacterial or a DNA vector. A bacterial vector may comprise recombinant Salmonella, recombinant Shigella or recombinant BCG. A DNA vector may comprise plasmid DNA.

Viruses or bacteria that are non- replicating or replication impaired may have arisen naturally or may have been produced artificially, for example, by genetic manipulation.

Preferably the one or more antigens encoded by one or more vectors are derived from a pathogen, such as a virus, a bacterium or a fungus. For example, the antigen encoded by the vector may be a protein or polypeptide or epitope derived from one or more of the following pathogens, HIV type 1 and 2 (HIV-1 and HIV-2 respectively), Human T Cell Leukaemia Virus types 1 and 2 (HTLV-1 and HTLV-2 respectively), Herpes Simplex Virus types 1 and 2 (HSV-1 and

HSV-2 respectively), a picornavirus, a hepadnavirus, a flavivirus, Haemophilus influenzae, human papilloma virus, Lassa fever virus, HBV, HCV, CBV, CMV, foot and mouth disease virus, Epstein Barr virus, Treponema pallidum, Neisseria gonorrhoea, a Plasmodium, a pathogenic Steptococcus species, M. Tuberculosis, Chlamydia trachomatis, Toxoplasma gondii, Leishmania species, Cytomegalovirus and Candida albicans. Alternatively, or additionally, the antigen may be a cancer antigen or epitope, a measles, mumps and/or rubella antigen or epitope, a tetanus antigen or epitope, a diphtheria antigen or epitope, a hepatitis A, B or C antigen or epitope, a polio antigen or epitope and/or an antigen or epitope relating to any other disease.

The one or more antigens or epitopes may be derived from bacteria selected from the list comprising mycobacteria, pneumococci, meningococci, Burkholderia, shigella and salmonella.

The one or more antigens or epitopes may be derived from an organism that causes a parasitic disease, this includes plasmodia, theileria, schistosomes, leishmania, eimeria.

A cancer antigen or epitope may include or be part of a human heat shock protein or a tumour associated antigen, such as, CEA, PSA, Muc 1 or Her2neu. The cancer antigen may be derived from one of the following cancers, lung, breast, kidney, colon, rectum, bone, brain, thyroid and haematological malignancies such as leukaemias and lymphomas

The antigen may be naturally expressed by the vector. For example, if the vector is an adenovirus, the antigen may be an adenovirus protein which may confer immunity against subsequent infection and/or disease caused by an adenovirus of the same or similar strain. Alternatively, or additionally, the antigen may be exogenous to the vector.

The vector may encode one or more antigens. If the vector encodes more than one antigen, the antigens may be derived from the same pathogen or disease, or from different pathogens or diseases.

The one or more antigens may be encoded on the same or different vectors. The one ore more antigens may be encoded on the same or different vectors to that which encode one or more TLR signalling pathway component.

The method of the invention may be used to immunise against diseases in which T cell responses play a protective role. Such diseases include, but are not limited to, malaria, infection and/or disease caused by the viruses HIV, herpes simplex, herpes zoster, hepatitis C, hepatitis B, influenza, Epstein Barr virus, measles, dengue, and HTLV-1; infection and/or disease caused by the bacterium Mycobacterium tuberculosis and Listeria spp; infection and/or disease caused by encapsulated bacteria such as streptococcus and haemophilus; infection and/or disease caused by parasites such as Leishmania, Toxoplasma and Trypanosoma. The method of the invention may also be used to immunise against certain forms of cancer, for example, melanoma, lymphomas and leukaemias, cancers of the lung, breast and colon, or against other diseases.

Preferably the immunogenic or vaccine composition is for use in therapeutic or prophylactic treatments or both.

The immune response elicited by any method of the invention may be therapeutic or prophylactic or both.

Wherein the one or more antigen and/or the one or more TLR signalling pathway component is encoded on two or more vectors, the vectors may be administered simultaneously or sequentially. For example, in one embodiment an antigen may be encoded on a first adenovirus vector and an adjuvant, such as a TLR signalling pathway component, may be encoded on a second adenovirus vector, the first and second adenovirus vectors may be administered to an organism simultaneously or sequentially, wherein sequential administration requires the two vectors to be administered within 3 days of each other, preferably within 2 day, more preferably within 24 hours. Simultaneous administration, or administration at the same time, preferably means the vectors are administered within at least two hours of each other. The two or more vectors may be administered at the same site, ie in the same arm, or at different sites, in one arm and in one leg. The two vectors may be administered by the same or different routes.

Where two or more vectors are used as a mixture, the vectors may be administered at the same or different times. Where two or more types of vector are used, each antigen is encoded on only one type of vector.

An immunogenic or vaccine composition according to the invention may be for oral, systemic, parenteral, topical, mucosal, intramuscular, intravenous, intraperitoneal, intradermal, subcutaneous, intranasal, intravaginal, intrarectal, transdermal, sublingual, inhalation or aerosol administration.

A composition according to the invention may be administered to a subject/organism in the form of a pharmaceutical composition. In addition to the immunogenic or vaccine composition, a pharmaceutical composition preferably comprises one or more physiologically and/or pharmaceutically effective carriers, diluents, excipients or auxiliaries which facilitate processing and/or delivery of the antigen and/or adjuvant.

Determination of an effective amount of an immunogenic or vaccine composition for administration to an organism is well within the capabilities of those skilled in the art. For example, for mouse to humans, a DNA vaccination dose may comprise from about 0.1 μg to about 10 mg. For an adenoviral vector the vaccination dose may be between about 1×10⁶ and 1×10¹⁶ viral particles per animal. For an MVA vector the vaccination dose may be between about 1×10² and 1×10¹⁰ pfu per animal.

In addition to the TLR signalling pathway component a further molecular adjuvant may be used. The further molecular adjuvant may be on the same vector as the TLR signalling pathway component and/or the one or more target antigens, or it may be on another vector. The further molecular adjuvant may be a co-stimulatory molecule or a cytokine, such as, IL7 or IL15.

A composition according to the invention may be administered with a non-vectored adjuvant.

A composition according to the invention may be used in isolation, or it may be combined with one or more other immunogenic or vaccine compositions, and/or with one or more other therapeutic regimes.

Preferably a TLR signalling pathway component which may be used as a molecular adjuvant can be identified by its ability to induce MIP2 expression when transfected in a suitable vector into RAW cells. Also, suitable combinations of TLR pathway components to be used together as adjuvants may be identified by their additive or synergistic induction of MIP2 expression when co-transfected into RAW 264.7 cells. Methods of transfecting RAW cells and using, for example luciferase expressing reported constructs with the MIP2 promoter, to determine MIP2 expression are known to those skilled in the art (see Dower and Kiss-Toth, 2002, Current Genomics 3: 139-148).

According to a further aspect the invention provides a kit for use in inducing an immune response in an organism, comprising an immunogenic or vaccine composition according to the invention and instructions relating to administration.

According to a yet further aspect, the invention provides a pharmaceutical composition comprising an immunogenic or a vaccine composition according to the invention and one or more physiologically effective carriers, diluents, excipients or auxiliaries.

According to another aspect, the invention provides the use of an immunogenic composition according to the invention in the preparation of a medicament for the treatment and/or prevention of infection and/or disease related to the antigen encoded by the vector in the immunogenic composition.

Where the antigen encoded by the vector in the composition is from a pathogen, the medicament may be intended/used to confer protection from infection and/or from disease caused by the pathogen from which the antigen of interest is derived. Alternatively, where the antigen encoded by the vector in the composition is a cancer antigen or an antigen associated with a particular disease, the medicament may be intended/used to confer protection from, and/or to treat, the cancer or the particular disease from which the antigen is derived.

The medicament or composition may be a vaccine.

According to another aspect the invention provides the use of an immunogenic composition according to the invention in the treatment and/or prevention of infection or disease related to the antigen encoded by a vector in the immunogenic composition.

Preferably, in a use according to the invention the composition or medicament induces an immune response when administered to an organism.

In an alternative embodiment of the invention the gene encoding the molecular adjuvant and the gene encoding the antigen may be provided on different vectors. Preferably, at least one of the vectors is a DNA vector, an adenovirus vector or an MVA vector.

The skilled man will appreciate that all preferred feature of the invention described with reference to only some aspects of the invention can be applied to all aspects of the invention.

Preferred embodiments of the present invention will now be described, merely by way of example, with reference to the following drawings and examples.

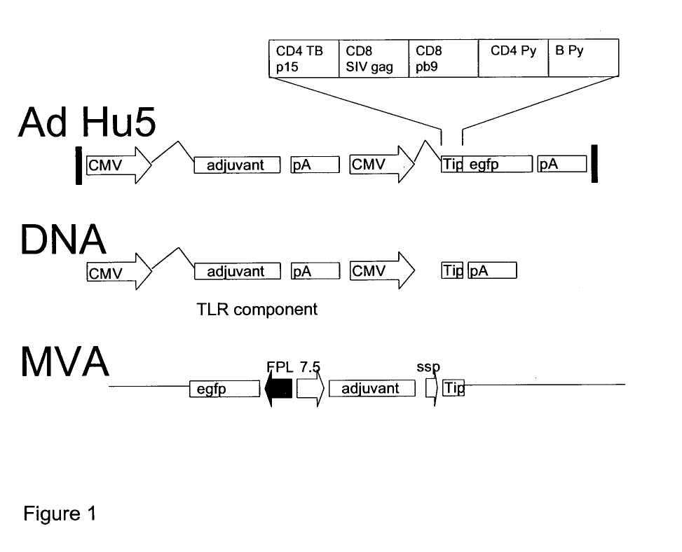

FIG. 1—is a schematic diagram illustrating the design of vectors used in the following examples. Essentially, this figure depicts schematically the adjuvant and antigen coding regions together with the relevant transcriptional control regions used in the adenovirus vector, the DNA vector and the MVA vector respectively as used in the following examples. “CMV” refers to the human CMV immediate-early gene promoter. “adjuvant” refers to the location of molecular adjuvants. “pA” refers to the poly adenylation site. “TIP” refers to an epitope string containing TB, immunodeficiency and Plasmodial epitopes;

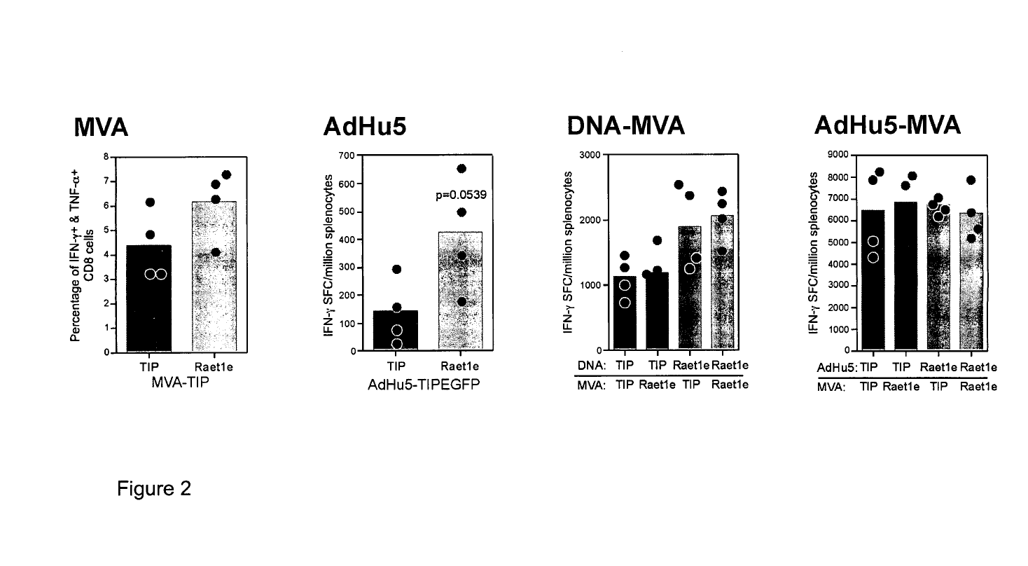

FIG. 2—Demonstrates the adjuvant effect of Raet1e. More specifically, FIG. 2 compares the effect of Raet1e on the immunogenicity of the CD8 epitope (Pb9) in the TIP string by comparing adjuvanted and unadjuvanted vectors. Taking each graph in turn from left to right:

MVA—illustrates the effect of the adjuvant Raet1e on the immunogenicity of the Pb9 epitope when delivered to mice in an MVA vector. The data depicts the percentage of IFN-γ and TNFα double positive Pb9 specific CD8 cells in the spleen 1 week after (i) vaccination with an MVA vector expressing the TIP epitope string and the adjuvant Raet1e (Raet1e), and (ii) vaccination with a control MVA vector expressing the TIP epitope string and no Raet1e (TIP).

AdHu5—illustrates the effect of the adjuvant Raet1e on the immunogenicity of the Pb9 epitope when delivered to mice in an AdHu5 (adenovirus) vector. The data depicts the numbers of Pb9 Interferon-γ positive splenocytes per million splenocytes studied. The splenocytes were assessed by ELIspot 2 weeks after vaccination with either (i) with 1×10⁸ AdHu5 expressing TIP-EGFP and Raet1e (Raet1e), or (ii) a control adenovirus expressing TIP-EGFP with no Raet1e (TIP).

DNA-MVA—illustrates the effect of the adjuvant Raet1e on the immunogenicity of the Pb9 epitope when delivered to mice in a DNA-MVA prime boost regime. Mice were vaccinated with a 50 mcg plasmid expressing TIPEGFP, together with either Raet1e (DNA: Raet1e) or a no adjuvant control (DNA:TIP). 2 weeks later, the mice received 1×10⁶ control MVA vectors without an adjuvant (MVA:TIP) or 1×10⁶ Raet1e adjuvanted MVA vector which also expressed TIPEGFP (MVA: Raet1e). The data depicts the numbers of Pb9 Interferon-γ positive splenocytes per million studied. The splenocytes were assessed by ELIspot 2 weeks following the MVA boost.

AdHu5-MVA—illustrates the effect of Raet1e on the immunogenicity of the Pb9 epitope when delivered to mice in a AdHu5-MVA prime-boost regime. Mice were vaccinated with 1×10⁸ AdHu5 expressing TIPEGFP, together with either Raet1e (AdHu5: Raet1e) or no adjuvant control (AdHu5:TIP). 8 weeks later, mice received 1×10⁶ control MVA vector with no adjuvant (MVA:TIP) or 1×10⁶ Raet1e adjuvanted MVA also expressing TIPEGFP (MVA: Raetle). The data depicts the numbers of Pb9 Interferon-γ positive splenocytes per million studied. The splenocytes were assessed by ELIspot 2 weeks following the MVA boost.

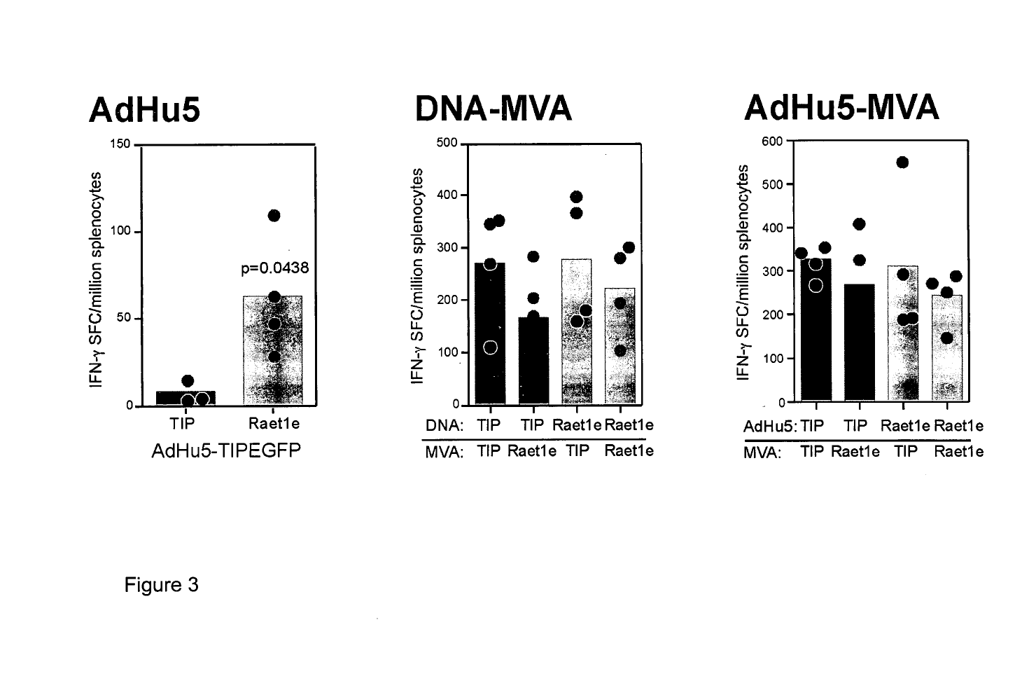

FIG. 3—Demonstrates the adjuvant effect of Raet1e. More specifically, FIG. 3 is a comparison of the effect of Raet1e adjuvanted with unadjuvanted vectors on the immunogenicity of the CD4 epitope (P15). Taking each graph in turn from left to right:

AdHu5—illustrates the effect of the adjuvant Raet1e on the immunogenicity of the P15 epitope when delivered to mice in an AdHu5 vector: The data depicts the number of P15 Interferon-γ positive splenocytes per million splenocytes studied. The splenocytes were assessed by ELIspot 2 weeks after (i) vaccination with 1×10⁸ AdHu5 expressing TIP-EGFP and Raet1e (Raetle), or (ii) vaccination with a control adenovirus expressing TIP-EGFP but no Raet1e (TIP).

DNA-MVA—illustrates the effect of the adjuvant Raet1e on the immunogenicity of the P15 epitope when delivered to mice in a DNA-MVA prime boost regime. Mice were vaccinated with a 50 mcg plasmid expressing TIPEGFP, together with either Raet1e (DNA: Raet1e) or a no adjuvant control (DNA:TIP). 2 weeks later, the mice received 1×10⁶ control (MVA:TIP) or Raet1e adjuvanted MVA also expressing TIPEGFP (MVA: Raet1e). The data depicts the numbers of P15 Interferon-γ positive splenocytes per million studied. The splenocytes were assessed by ELIspot 2 weeks following the MVA boost.

AdHu5-MVA—illustrates the effect of Raet1e on the immunogenicity of the P15 epitope when delivered to mice in an AdHu5-MVA prime-boost regime. Mice were vaccinated with 1×10⁸ AdHu5 expressing TIPEGFP, together with either Raet1e (AdHu5: Raet1e) or no adjuvant control (AdHu5:TIP). 8 weeks later, mice received 1×10⁶ control (MVA:TIP) or Raet1e adjuvanted MVA also expressing TIPEGFP (MVA: Raet1e). The data depicts the numbers of P15 Interferon-γ positive splenocytes per million studied. The splenocytes were assessed by ELIspot 2 weeks following the MVA boost.

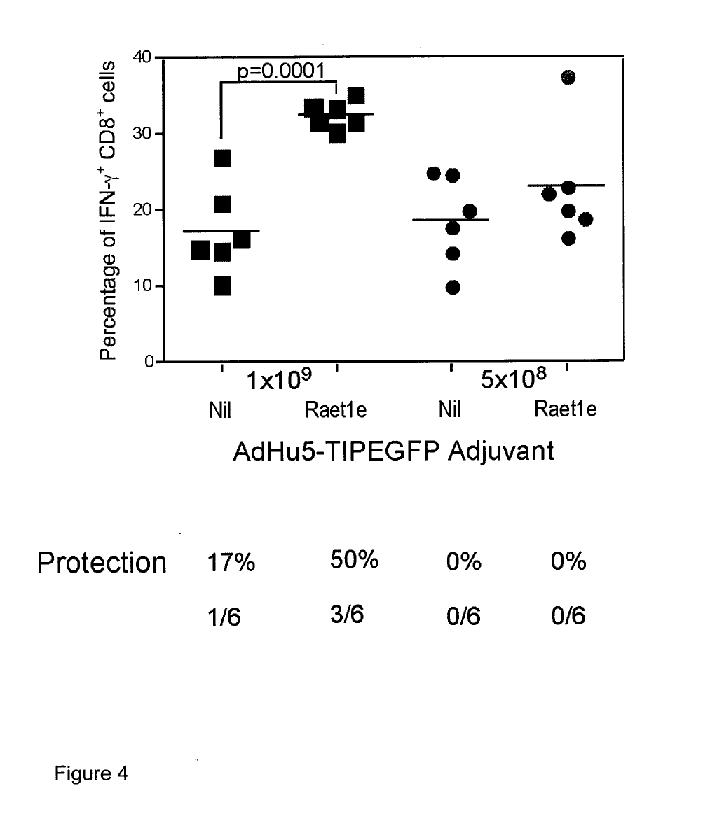

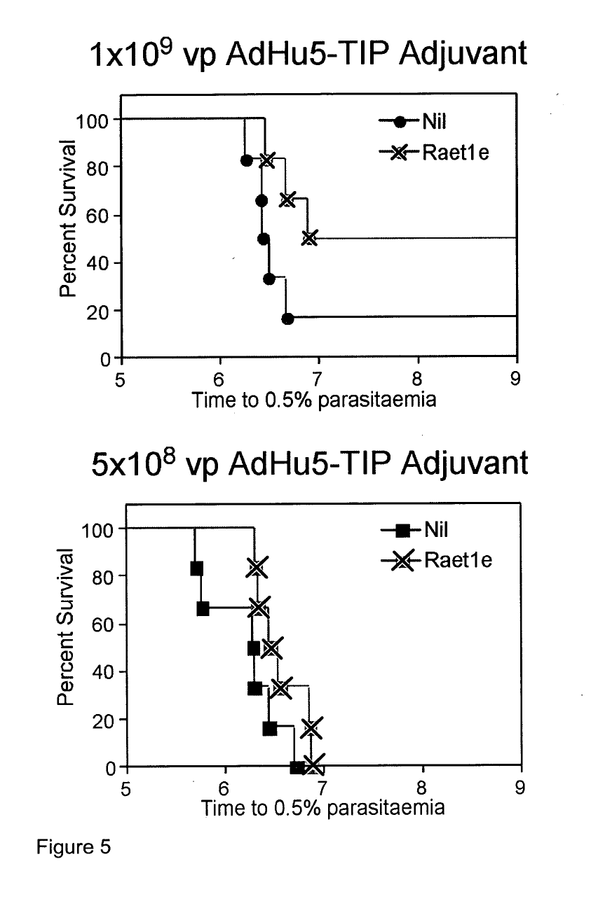

FIG. 4—illustrates that mice immunised with adenoviruses expressing the molecular adjuvant Raet1e have enhanced numbers of interferon-gamma specific cells in the blood, compared with control adenoviruses. FIG. 4 shows the responses two weeks after vaccination with 1×10⁹ (left) or 5×10⁸ (right) of adenovirus expressing TIPEGFP and either no adjuvant (control) or Raet1e. The day after this analysis was performed on blood, mice were challenged with 1,000 Plasmodium berghei sporozoites. The survival curves are shown in FIG. 5.

FIG. 5—Demonstrates that the use of Raet1e as an adjuvant increases the survival of immunised animals, thereby illustrating the protective efficacy of vectored vaccines according to the invention. The animals shown in FIG. 4 were challenged with 1,000 P berghei sporozoites intravenously and the emergence of parasites in the blood was determined by blood smear analysis as described in the methods section. Kaplan-Meier survival curves are shown;

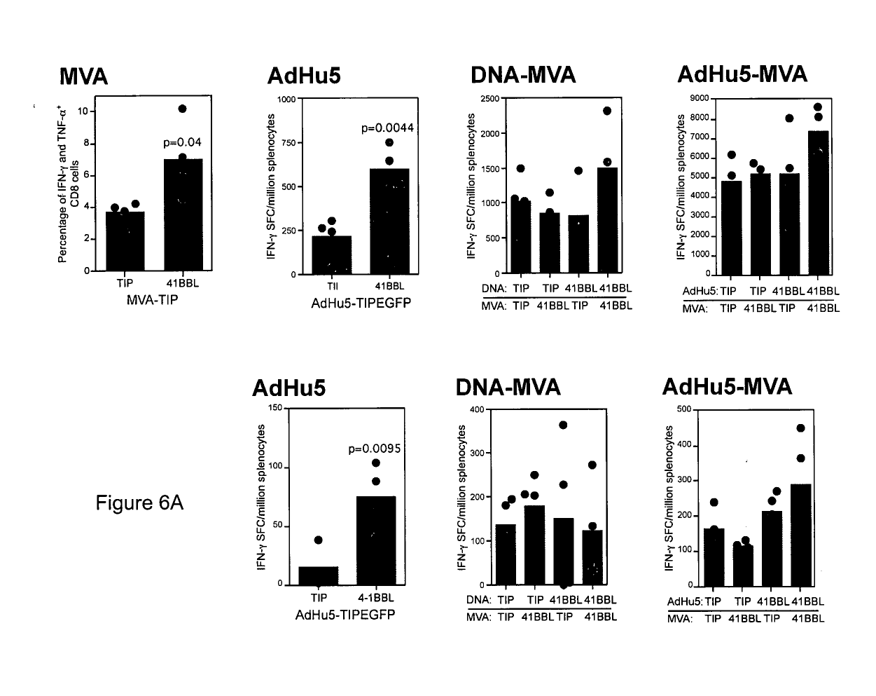

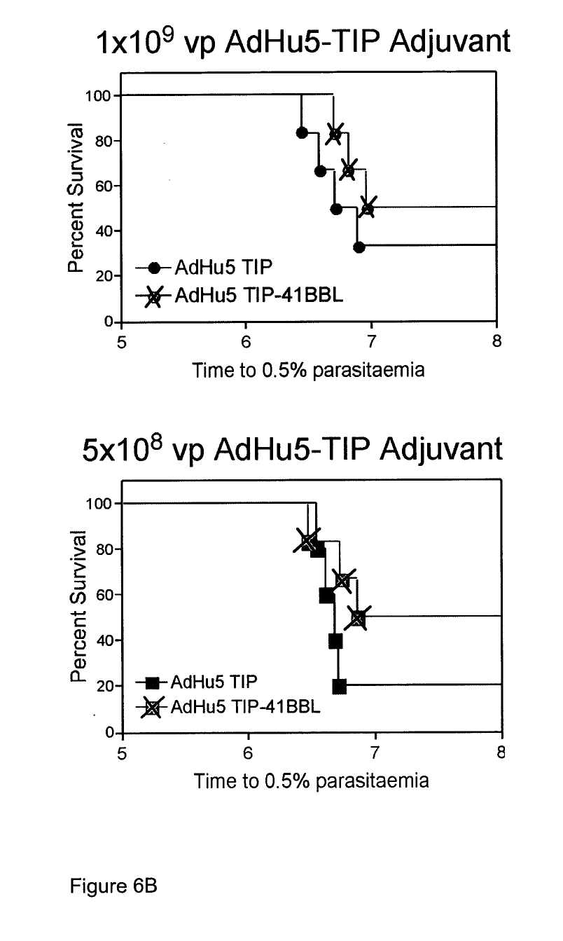

FIG. 6A—Demonstrates the adjuvant effect of 4-1BBL. More specifically, the upper four figures in FIG. 6 compare the effect of 4-1BBL on the immunogenicity of the CD8 epitope in the TIP sequence (Pb9) by comparing adjuvanted and unadjuvanted vectors. Taking each graph in turn from left to right:

MVA—illustrates the effect of the adjuvant 4-1BBL on the immunogenicity of the Pb9 epitope when delivered to mice in an MVA vector. The data depicts the percentage of IFN-γ and TNFα double positive Pb9 specific CD8 cells in the spleen 1 week after (i) vaccination with an MVA vector expressing the TIP epitope string and the adjuvant 4-1BBL (41BBL), and (ii) vaccination with a control MVA vector expressing the TIP epitope string and no 4-1BBL (TIP).

AdHu5—illustrates the effect of the adjuvant 4-1BBL on the immunogenicity of the Pb9 epitope when delivered to mice in an AdHu5 vector. The data depicts the numbers of Pb9 Interferon-γ positive splenocytes per million splenocytes studied. The splenocytes were assessed by ELIspot 2 weeks after vaccination with either (i) with 1×10⁸ AdHu5 expressing TIP-EGFP and 4-1BBL (41BBL), or (ii) a control adenovirus expressing TIP-EGFP with no 4-1BBL (TIP).

DNA-MVA—illustrates the effect of the adjuvant 4-1BBL on the immunogenicity of the Pb9 epitope when delivered to mice in a DNA-MVA prime boost regime. Mice were vaccinated with a 50 mcg plasmid expressing TIPEGFP, together with either 4-1BBL (DNA: 41BBL) or a no adjuvant control (DNA:TIP). 2 weeks later, the mice received 1×10⁶ control vector (MVA:TIP) without an adjuvant or 4-1BBL adjuvanted MVA vector also expressing TIPEGFP (MVA:41BBL). The data depicts the numbers of Pb9 Interferon-65 positive splenocytes per million studied. The splenocytes were assessed by ELIspot 2 weeks following the MVA boost.

AdHu5-MVA—illustrates the effect of 4-1BBL on the immunogenicity of the Pb9 epitope when delivered to mice in a AdHu5-MVA prime-boost regime. Mice were vaccinated with 1×10⁸ AdHu5 expressing TIPEGFP, together with either 4-1BBL (AdHu5:41BBL) or no adjuvant control (AdHu5:TIP). 8 weeks later, mice received 1×10⁶ control (MVA:TIP) or 4-1BBL adjuvanted MVA also expressing TIPEGFP (MVA:41BBL). The data depicts the numbers of Pb9 Interferon-65 positive splenocytes per million studied. The splenocytes were assessed by ELIspot 2 weeks following the MVA boost.

The lower 3 figures in FIG. 6 is a comparison of the effect of 4-1BBL adjuvanted with unadjuvanted vectors on the immunogenicity of the CD4 epitope (P15). Taking each graph in turn from left to right:

AdHu5—illustrates the effect of the adjuvant 4-1BBL on the immunogenicity of the P15 epitope when delivered to mice in an AdHu5 vector: The data depicts the number of P15 Interferon-γ positive splenocytes per million splenocytes studied. The splenocytes were assessed by ELIspot 2 weeks after (i) vaccination with 1×10⁸ AdHu5 expressing TIP-EGFP and 4-1BBL (41BBL), or (ii) vaccination with a control adenovirus expressing TIP-EGFP but no 4-1BBL (TIP).

DNA-MVA—illustrates the effect of the adjuvant 4-1BBL on the immunogenicity of the P15 epitope when delivered to mice in a DNA-MVA prime boost regime. Mice were vaccinated with a 50 mcg plasmid expressing TIPEGFP, together with either 4-1BBL (DNA:41BBL) or a no adjuvant control (DNA:TIP). 2 weeks later, the mice received 1×10⁶ control (MVA:TIP) or 4-1BBL adjuvanted MVA also expressing TIPEGFP (MVA:41BBL). The data depicts the numbers of P15 Interferon-γ positive splenocytes per million studied. The splenocytes were assessed by ELIspot 2 weeks following the MVA boost.

AdHu5-MVA—illustrates the effect of 4-1BBL on the immunogenicity of the P15 epitope when delivered to mice in aN AdHu5-MVA prime-boost regime. Mice were vaccinated with 1×10⁸ AdHu5 expressing TIPEGFP, together with either 4-1BBL (AdHu5:41BBL) or no adjuvant control (AdHu5:TIP). 8 weeks later, mice received 1×10⁶ control (MVA:TIP) or 4-1BBL adjuvanted MVA also expressing TIPEGFP (MVA:41BBL). The data depicts the numbers of P15 Interferon-γ positive splenocytes per million studied. The splenocytes were assessed by ELIspot 2 weeks following the MVA boost.

FIG. 6B—Demonstrates that the use of 4-1BBL as an adjuvant increases the survival of immunised animals, thereby illustrating the protective efficacy of vectored vaccines according to the invention. The animals shown in FIG. 6 were challenged with 1,000 P berghei sporozoites intravenously and the emergence of parasites in the blood was determined by blood smear analysis as described in the methods section. Kaplan-Meier survival curves are shown;

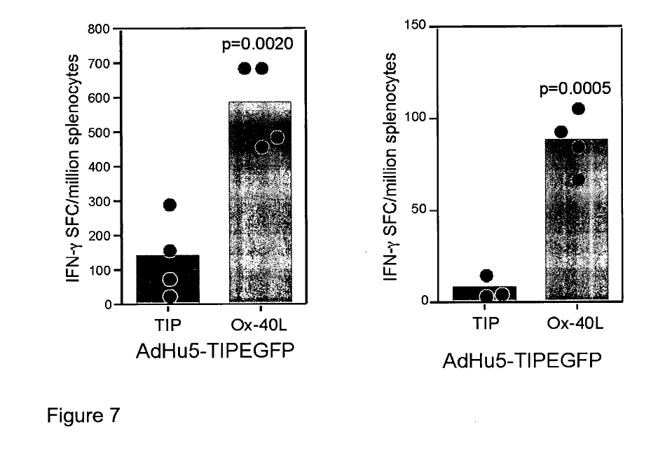

FIG. 7—Demonstrates the adjuvant effect of OX4OL. More specifically, the figure compares the effect of OX4OL on the immunogenicity of the CD8 epitope (Pb9) in the left hand graph and the CD4 epitope (P15) in the right hand graph, by comparing adjuvanted and unadjuvanted vectors.

AdHu5—(the left hand figure) illustrates the effect of the adjuvant OX4OL on the immunogenicity of the Pb9 epitope when delivered to mice in an AdHu5 vector. The data depicts the numbers of Pb9 Interferon-γ positive splenocytes per million splenocytes studied. The splenocytes were assessed by ELIspot 2 weeks after vaccination with either (i) with 1×10⁸ AdHu5 expressing TIP-EGFP and OX40L (OX40L), or (ii) a control adenovirus expressing TIP-EGFP with no OX40L (TIP).

AdHu5—(the right hand figure) illustrates the effect of the adjuvant OX40L on the immunogenicity of the P15 epitope when delivered to mice in an AdHu5 vector: The data depicts the number of P15 Interferon-γ positive splenocytes per million splenocytes studied. The splenocytes were assessed by ELIspot 2 weeks after (i) vaccination with 1×10⁸ AdHu5 expressing TIP-EGFP and OX40L (OX40L), or (ii) vaccination with a control adenovirus expressing TIP-EGFP but no OX40L (TIP).

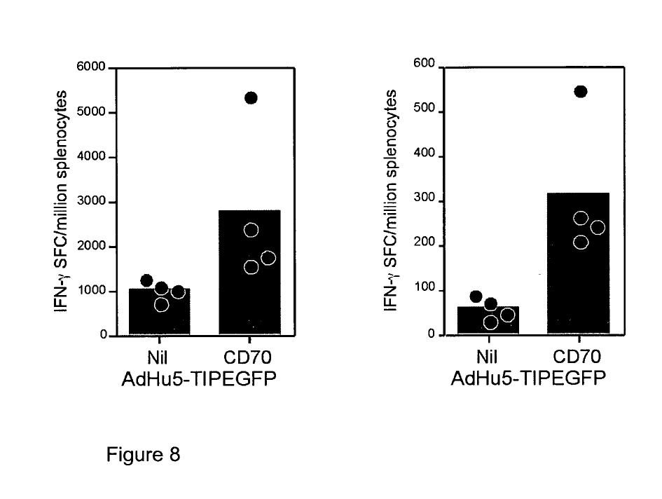

FIG. 8—Demonstrates the adjuvant effect of CD70. More specifically, the figure compares the effect of CD70 on the immunogenicity of the CD8 epitope (Pb9) in the left hand graph and the CD4 epitope (P15) in the right hand graph, by comparing adjuvanted and unadjuvanted vectors.

AdHu5—(the left hand figure) illustrates the effect of the adjuvant CD70 on the immunogenicity of the Pb9 epitope when delivered to mice in an AdHu5 vector. The data depicts the numbers of Pb9 Interferon-γ positive splenocytes per million splenocytes studied. The splenocytes were assessed by ELIspot 2 weeks after vaccination with either (i) with 1×10⁸ AdHu5 expressing TIP-EGFP and CD70 (CD70), or (ii) a control adenovirus expressing TIP-EGFP with no OX40L (TIP).

AdHu5—(the right hand figure) illustrates the effect of the adjuvant CD7 on the immunogenicity of the P15 epitope when delivered to mice in an AdHu5 vector: The data depicts the number of P15 Interferon-γ positive splenocytes per million splenocytes studied. The splenocytes were assessed by ELIspot 2 weeks after (i) vaccination with 1×10⁸ AdHu5 expressing TIP-EGFP and CD70 (CD70), or (ii) vaccination with a control adenovirus expressing TIP-EGFP but no CD70 (TIP).

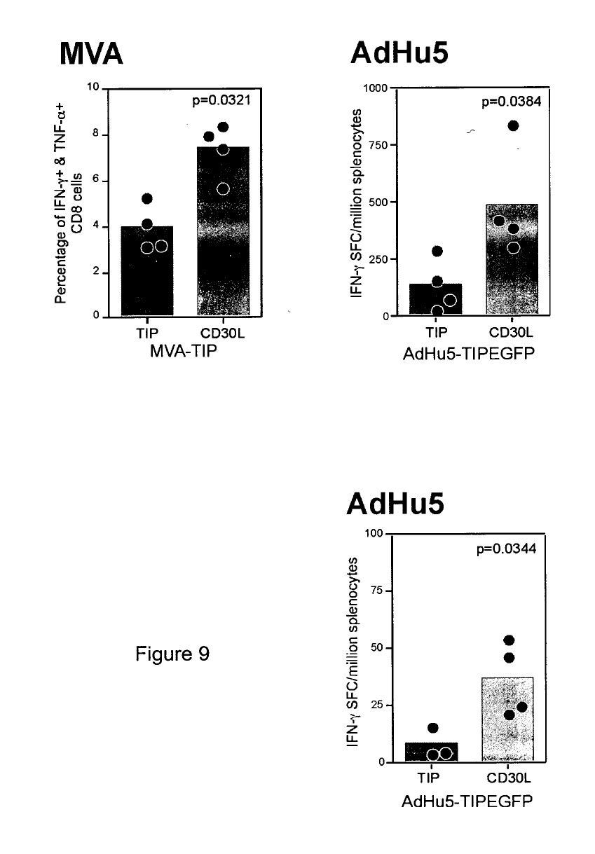

FIG. 9—Demonstrates the adjuvant effect of CD30L. More specifically, the upper two figures in FIG. 9 compare the effect of CD30L on the immunogenicity of the CD8 epitope (Pb9) and the lower figure compares the effect of CD30L on the immunogenicity of the CD4 epitope (P15), by comparing adjuvanted and unadjuvanted vectors. Taking each graph in turn from left to right:

MVA—(upper left figure) illustrates the effect of the adjuvant CD30L on the immunogenicity of the Pb9 epitope when delivered to mice in an MVA vector. The data depicts the percentage of IFN-γ and TNFα double positive Pb9 specific CD8 cells in the spleen 1 week after (i) vaccination with an MVA vector expressing the TIP epitope string and the adjuvant CD30L (CD30L), and (ii) vaccination with a control MVA vector expressing the TIP epitope string and no CD30L (TIP).

AdHu5—(upper right figure) illustrates the effect of the adjuvant CD30L on the immunogenicity of the Pb9 epitope when delivered to mice in an AdHu5 vector. The data depicts the numbers of Pb9 Interferon-γ positive splenocytes per million splenocytes studied. The splenocytes were assessed by ELIspot 2 weeks after vaccination with either (i) with 1×10⁸ AdHu5 expressing TIP-EGFP and CD30L (CD30L), or (ii) a control adenovirus expressing TIP-EGFP with no CD30L (TIP).

AdHu5—(lower figure) illustrates the effect of the adjuvant CD30L on the immunogenicity of the P15 epitope when delivered to mice in an AdHu5 vector: The data depicts the number of P15 Interferon-γ positive splenocytes per million splenocytes studied. The splenocytes were assessed by ELIspot 2 weeks after (i) vaccination with 1×10⁸ AdHu5 expressing TIP-EGFP and CD30L (CD30L), or (ii) vaccination with a control adenovirus expressing TIP-EGFP but no CD30L (TIP).

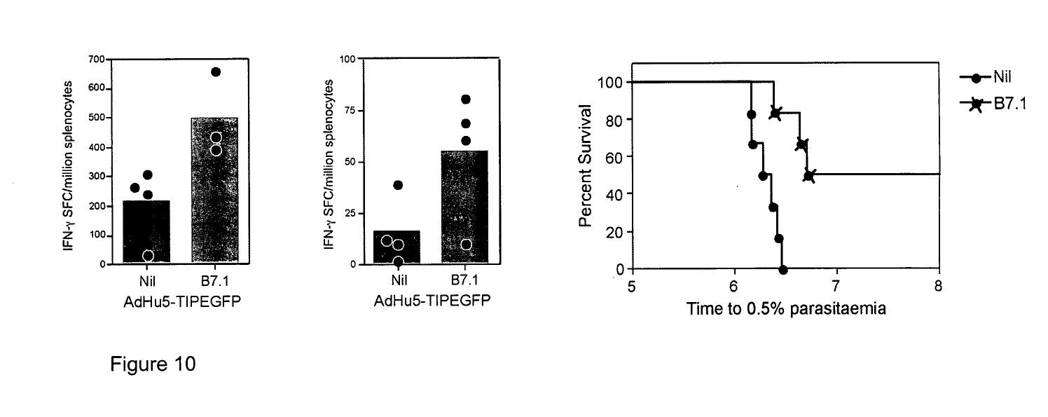

FIG. 10—Demonstrates the adjuvant effect of B7.1. More specifically, the left hand figure compares the effect of B7.1 on the immunogenicity of the CD8 epitope (Pb9) by comparing adjuvanted and unadjuvanted vectors. The middle hand figure compares the effect of B7.1 on the immunogenicity of the CD4 epitope (P15) by comparing adjuvanted and unadjuvanted vectors.

The right hand figure demonstrates that the use of B7.1 as an adjuvant increased the survival of immunised animals, thereby illustrating the protective efficacy of vectored vaccines according to the invention. The animals shown in FIG. 10 were challenged with 1,000 P berghei sporozoites intravenously and the emergence of parasites in the blood was determined by blood smear analysis as described in the methods section. Kaplan-Meier survival curves are shown;

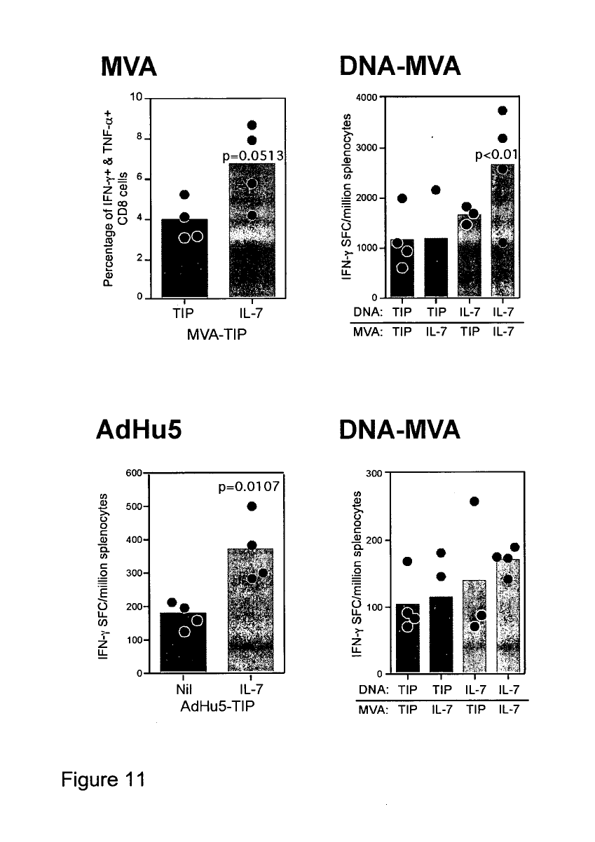

FIG. 11—demonstrates the adjuvant effect of IL-7. More specifically, FIG. 11 compares the effect of IL-7 on the immunogenicity of the CD8 epitope (Pb9) in the TIP string (upper graphs) and of the CD4 epitope (P15) in the TIP sting, by comparing adjuvanted and unadjuvanted vectors. Taking each graph in turn from left to right:

MVA—(upper left) illustrates the effect of the adjuvant IL-7 on the immunogenicity of the Pb9 epitope when delivered to mice in an MVA vector. The data depicts the percentage of IFN-γ and TNFα double positive Pb9 specific CD8 cells in the spleen 1 week after (i) vaccination with an MVA vector expressing the TIP epitope string and the adjuvant IL-7 (IL-7), and (ii) vaccination with a control MVA vector expressing the TIP epitope string and no IL-7 (TIP).

DNA-MVA—(upper right) illustrates the effect of the adjuvant IL-7 on the immunogenicity of the Pb9 epitope when delivered to mice in a DNA-MVA prime boost regime. Mice were vaccinated with a 50 mcg plasmid expressing TIPEGFP, together with either IL-7 (DNA:IL-7) or a no adjuvant control (DNA:TIP). 2 weeks later, the mice received 1×10⁶ control MVA vectors without an adjuvant (MVA:TIP) or 1×10⁶ IL-7 adjuvanted MVA vector which also expressed TIPEGFP (MVA:IL-7). The data depicts the numbers of Pb9 Interferon-γ positive splenocytes per million studied. The splenocytes were assessed by ELIspot 2 weeks following the MVA boost.

AdHu5—(lower left) illustrates the effect of the adjuvant IL-7 on the immunogenicity of the P15 epitope when delivered to mice in an AdHu5 vector: The data depicts the number of P15 Interferon-γ positive splenocytes per million splenocytes studied. The splenocytes were assessed by ELIspot 2 weeks after (i) vaccination with 1×10⁸ AdHu5 expressing TIP-EGFP and IL-7 (IL-7), or (ii) vaccination with a control adenovirus expressing TIP-EGFP but no IL-7 (TIP).

DNA-MVA—(lower right) illustrates the effect of the adjuvant IL-7 on the immunogenicity of the P15 epitope when delivered to mice in a DNA-MVA prime boost regime. Mice were vaccinated with a 50 mcg plasmid expressing TIPEGFP, together with either IL-7 (DNA:IL-7) or a no adjuvant control (DNA:TIP). 2 weeks later, the mice received 1×10⁶ control (MVA:TIP) or IL-7 adjuvanted MVA also expressing TIPEGFP (MVA:IL-7). The data depicts the numbers of P15 Interferon-γ positive splenocytes per million studied. The splenocytes were assessed by ELIspot 2 weeks following the MVA boost.

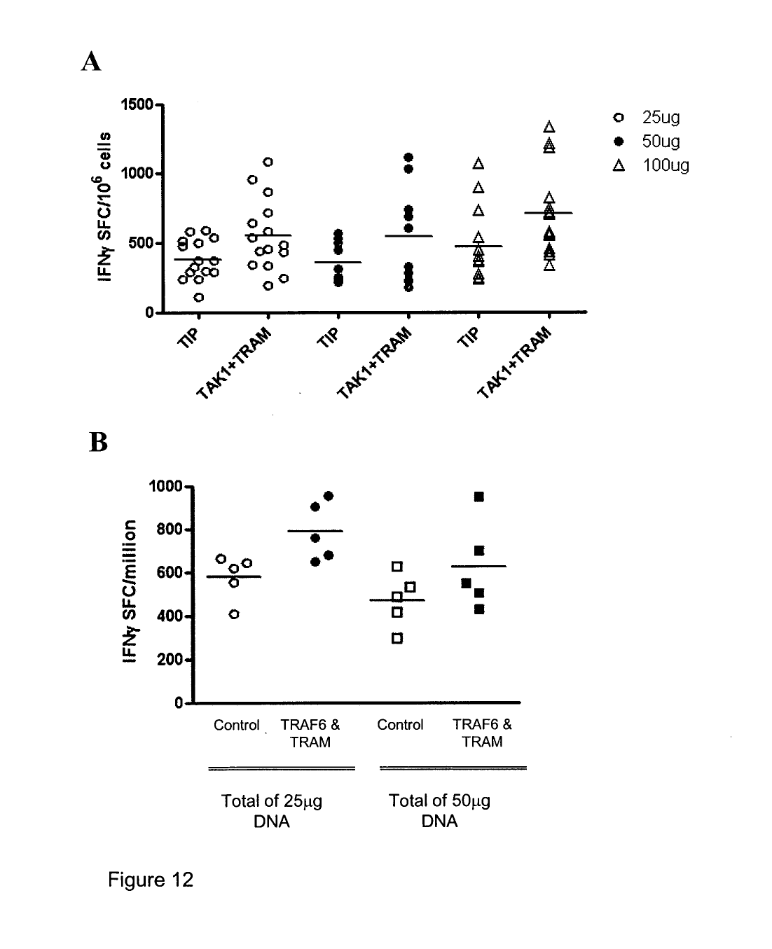

FIG. 12A—illustrates the adjuvant effect of TAK1 and TRAM when administered on separate vectors. Groups of 4-6 mice were vaccinated intramuscularly with separate plasmids. One group received a mixture of two plasmids, one co-expressing antigen (TIP) and TRAM, the other co-expressing antigen (TIP) and TAK1. The other group received a control plasmid, expressing antigen (TIP) but without adjuvants. The plasmid design is described in the methods. The plasmids were administered in multiple experiments at doses of 25, 50 and 100 mcg im. FIG. 11 shows the mean spleen Pb9 specific ELIspot responses 14 days after the second DNA immunization. Increases in immunogenicity are observed at all doses tested;

FIG. 12B—illustrates the adjuvant effect of TRAF6 and TRAM when administered on separate vectors. Groups of 5 mice were vaccinated intramuscularly with two plasmids. One group received one plasmid co-expressing antigen (TIP) and TRAM and one plasmid co-expressing antigen (TIP) and TRAF6. The other group received a control plasmid, expressing antigen (TIP) but without adjuvants. The plasmid design is described in the methods. The plasmids were administered in multiple experiments at doses of 25, and 50 mcg im. Spleen Pb9 specific ELIspot responses 14 days after the second DNA immunization are shown. An increase in immunogenicity was observed at all doses tested.

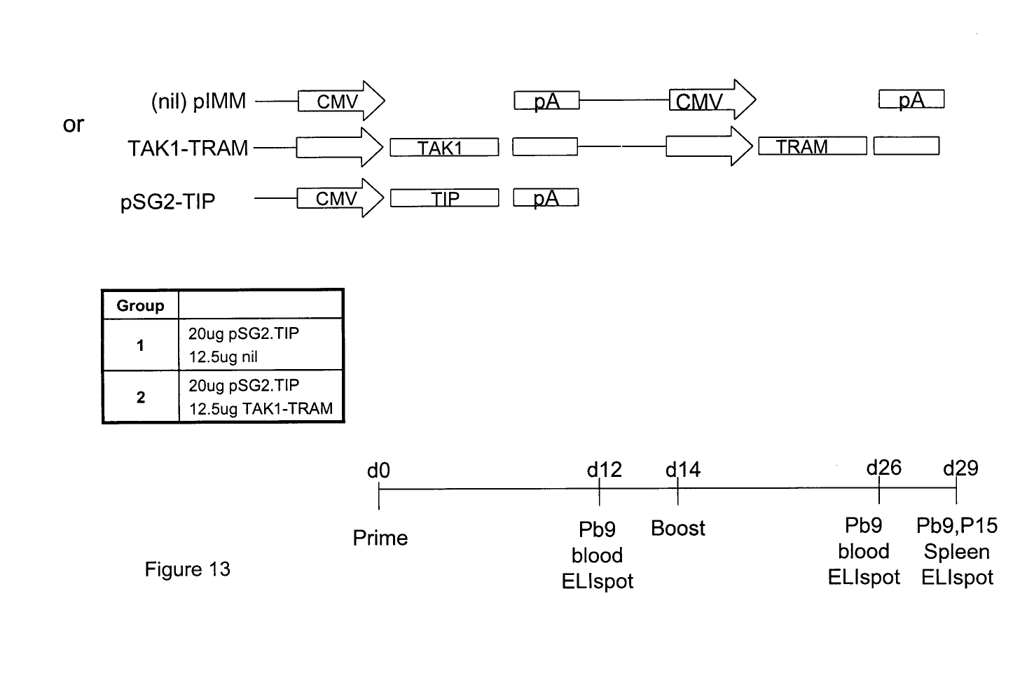

FIG. 13—illustrates the protocol used to investigate the adjuvant effect of TAK1 and TRAM when administered/co-expressed in the same vector. Mice were vaccinated with a combination of an antigen-expressing vector, pSG2.TIP or pSG2.HIVANP, and an adjuvant vector (expressing TAK1 and TRAM and the TIP antigen) in the amounts shown. The vector contained two expression cassettes, which encoded no protein (control) or encoded TAK1 and TRAM (test adjuvant vector). Vaccination intramuscularly and the assay followed the regime illustrated;

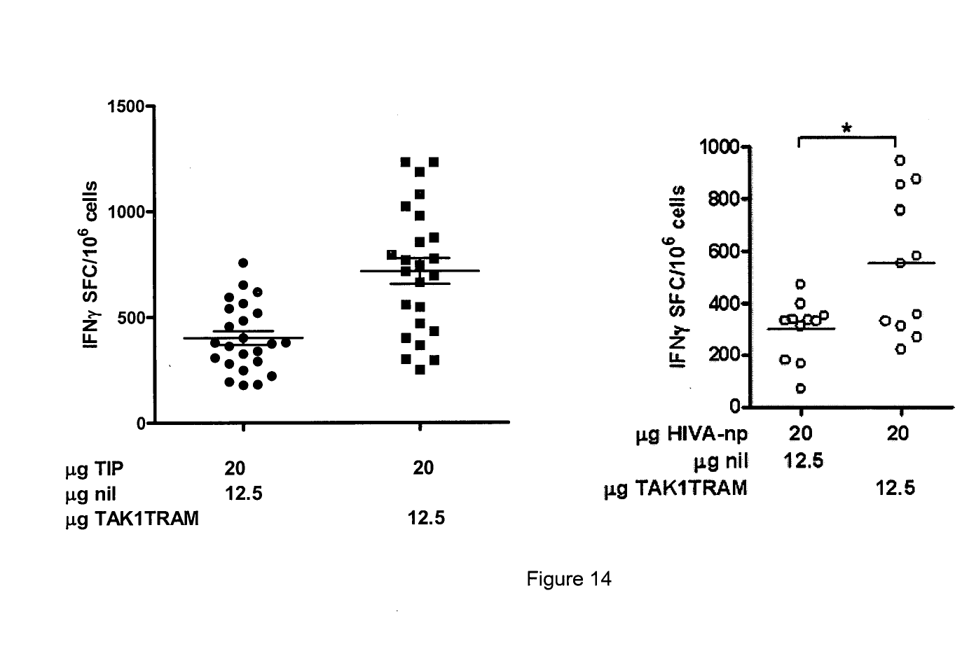

FIG. 14—illustrates the adjuvant effect of TAK1 and TRAM when administered in the same vector as described in FIG. 13. TAK1 and TRAM in combination in the same plasmid increase immunogenicity of DNA vectors: as assessed by spleen pb9 (CD8) specific IFN-γ specific cell numbers are TIP vaccination (left hand graph), and spleen epitope H (CD8) specific IFN-γ specific cell numbers are HIVANP vaccination (right hand graph);

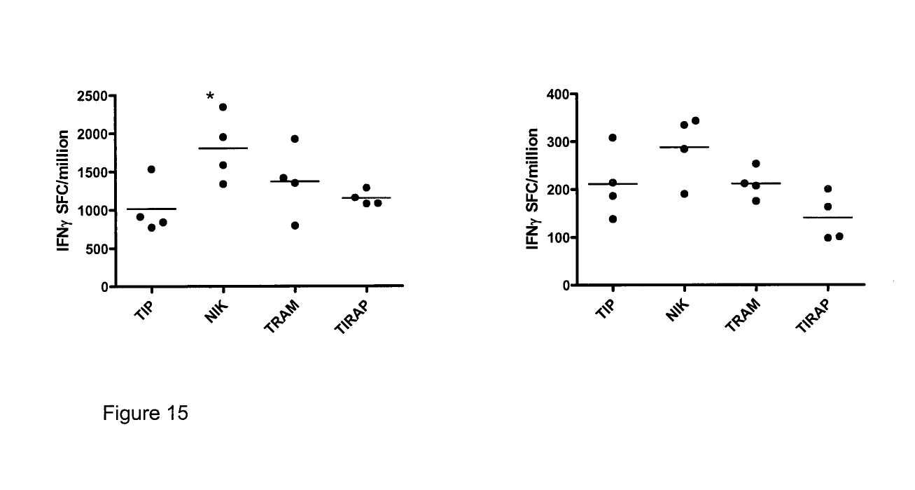

FIG. 15—illustrates the effect of TLR signaling pathway components on adenoviral immunogenicity. The numbers of Pb9 (CD8 epitope, left) and P15 (CD4 epitope, right) Interferon-γ splenocytes, were determined by ELIspot, 2 weeks following vaccination with 10⁸ AdHu5 expressing TIP-EGFP, either with no adjuvant (TIP), NIK, TRAM, or TIRAP;

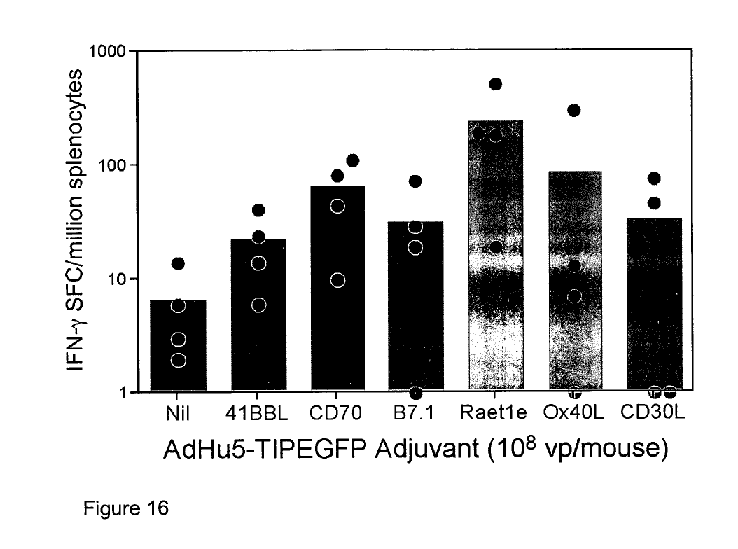

FIG. 16—illustrates the adjuvant effect of the co-stimulatory molecules 4-1BBL, CD70, B7.1, Raet1e, OX40L and CD30L on the immune response in mice to the SIV-gag antigen. Six mice were administered an adenovirus based vector (AdHu5) encoding the TIP antigen epitope and the adjuvant indicated. The number of spleen cells secreting SIV-gag specific IFN-γ were quantified after vaccination. All compounds tested showed an adjuvant effect.

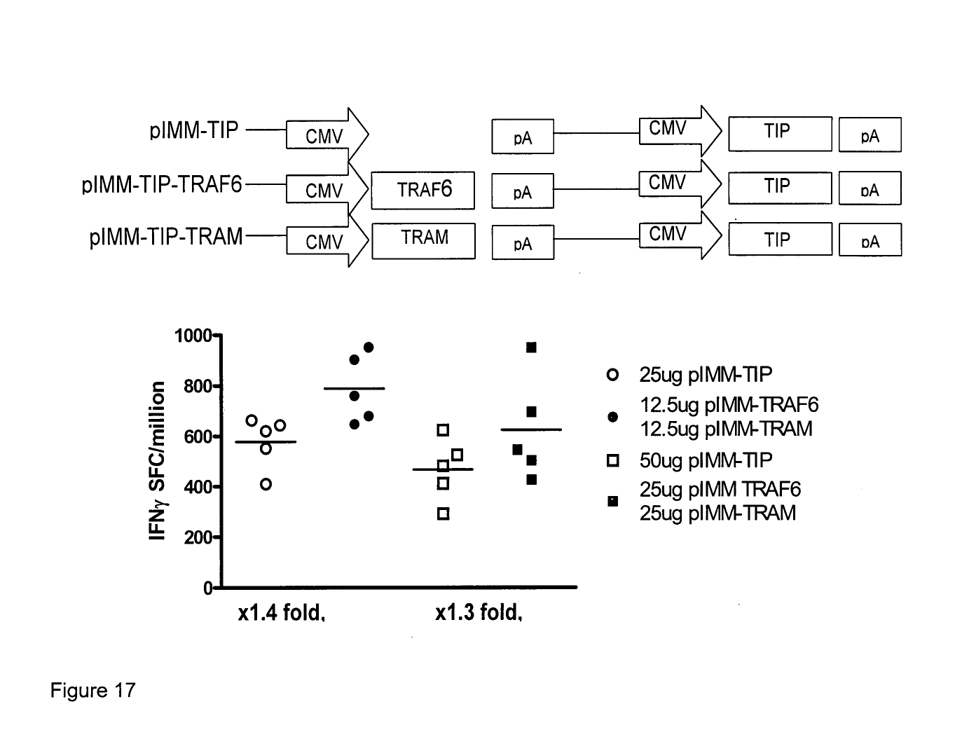

FIG. 17—illustrates the effect of TRAF6 and TRAM when administered on separate DNA vectors to mice. Mice were administered with either a plasmid encoding TIP only, or two plasmid, one encoding TRAF6 and one encoding TRAM. The effect of TRAF6 and TRAM on immunogenicity was determined by determining the number of spleen cells expressing Pb9 specific IFNγ after immunisation. The results show that the mixture of TRAM and TRAF6 have an effect, although this effect is quite small, only about 1.4 fold.

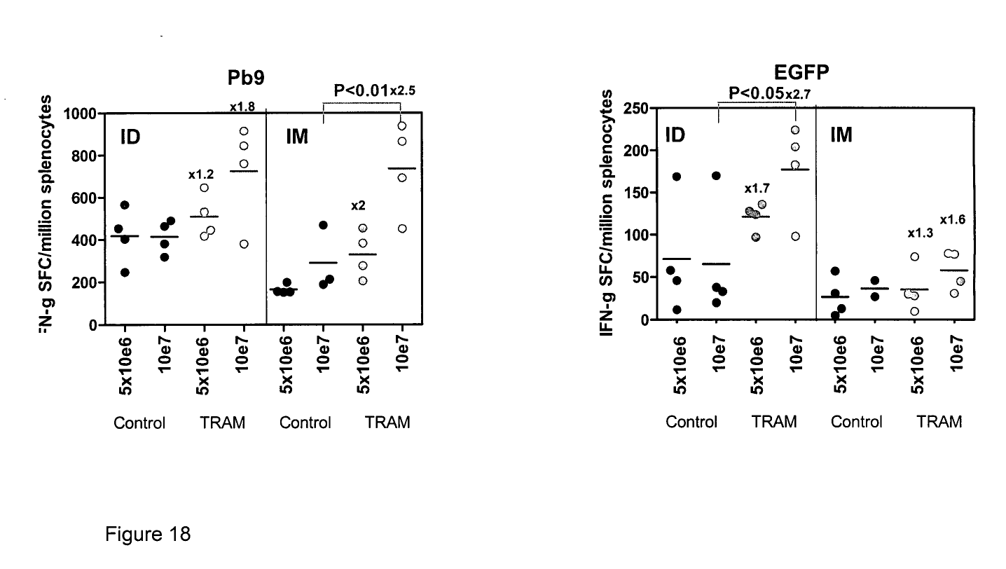

FIG. 18—illustrates the effect of expression of TRAM on adenoviral immunogenicity. Adenovirus Hu5 co-expressing murine TRAM, or no adjuvant molecule, and a TIP-EGFP fusion protein, were generated and purified by caesium chloride centrifugation. Viruses were injected at two doses either i.m. or i.d. and spleen CD8 T cell responses determined 14 weeks later. Significantly increased responses were elicited by AdHu5-TRAM-TIPEGFP compared with AdHu5-control-TIPEGFP (figure), when pb9 (left) and EGFP (CD9 epitope, right) Interferon-γ splenocyte numbers were determined by ELIspot.

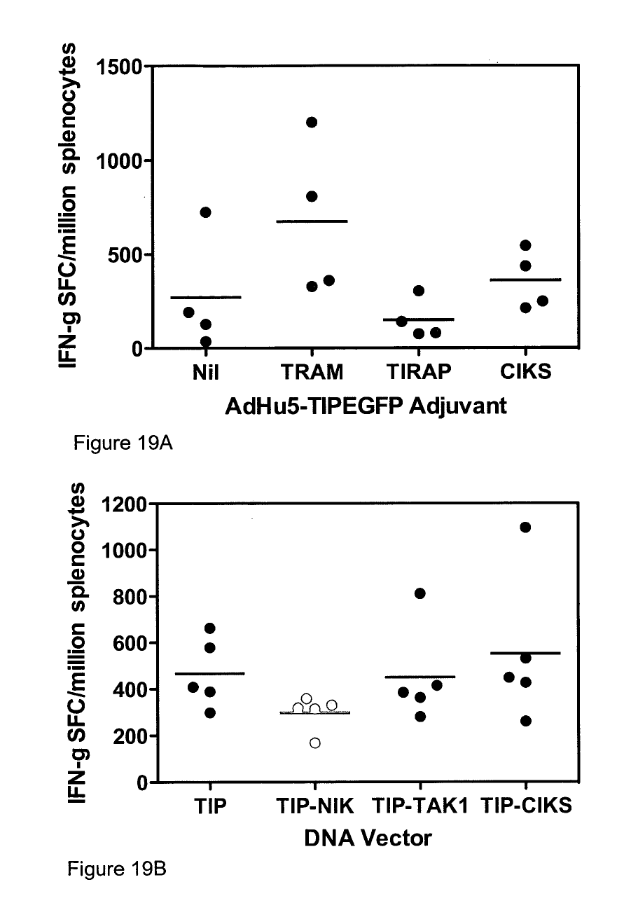

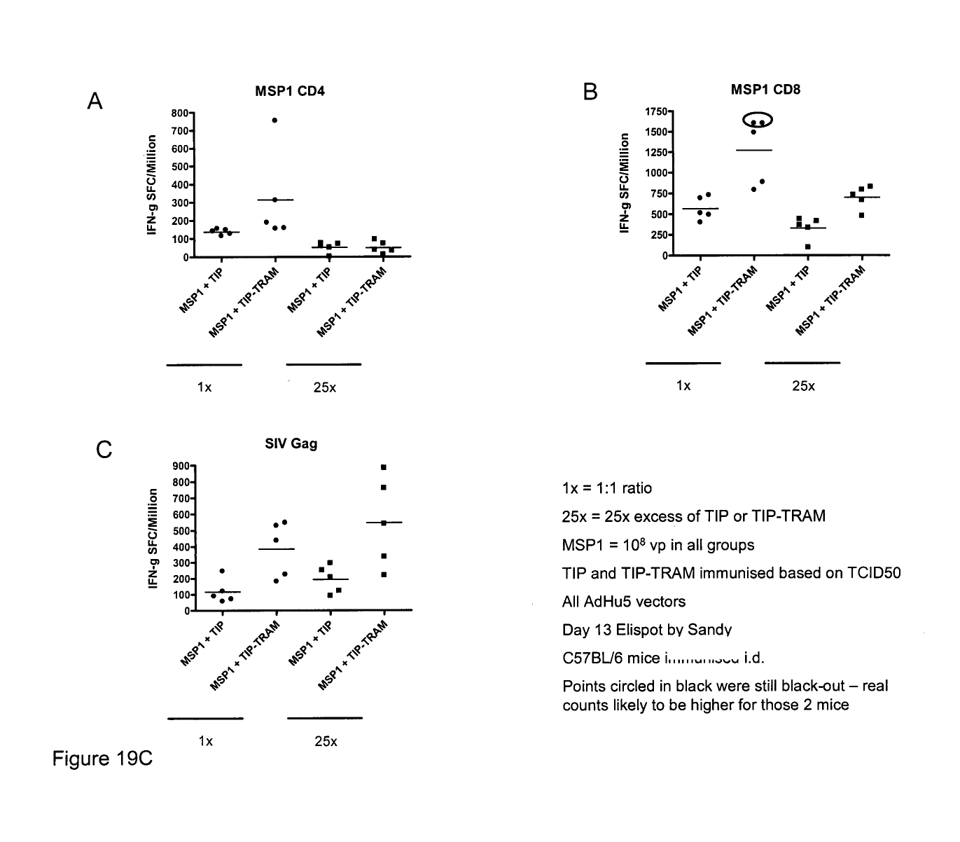

FIG. 19A and 19B—FIG. 19A illustrates the results of the use of an adenoviral vector encoding a TLR pathway component and the antigen TIPEGFP. Balb/c mice were immunised with 10⁷ vp of AdHuTIPEFGPadjuvant vector and a spleen ELIspot performed two weeks later to measure the antigen specific response to Pb9. TRAM here acts as an adjuvant in cis. FIG. 19B illustrates the results of the use of plasmid DNAs encoding TIP plus various TLR signalling molecules in the same plasmid vector to induce CD8 T cell responses in murine spleens to the TIP antigen. Balb/c mice were immunised with 50 μg of DNA in each immunisation and immunisations were carried out two weeks apart.

FIG. 19C—illustrates the ability of AdHu5-TIP-TRAM to adjuvant CD8 + T cell responses in trans. C57BL/6 mice were immunised as follows. All mice received 10⁸ vp AdHu5-PfM128 (known as AdHu5-MSP1) intradermally. This vaccine was mixed with either 1.5×10⁷ TCID50 infectious particles AdHu5-TIP or AdHu5-TIP-TRAM (1×), or 3.7×10⁸ TCID50 infectious particles AdHu5-TIP or AdHu5-TIP-TRAM (25×). 13 d later T cell responses were assessed in the spleen by ex-vivo IFN-gamma ELIspot following re-stimulation with peptide(s) corresponding to (A) a single CD4+ H-2b T cell epitope in MSP1, (B) four CD8 + H-2b T cell epitopes in MSP1 or (C) a single CD8 + H-2b T cell epitope from SIV-Gag present in the TIP epitope string. Mean responses and individual data points are shown as IFN-gamma spot form cells (SFC) per million splenocytes (n=5 mice per group).

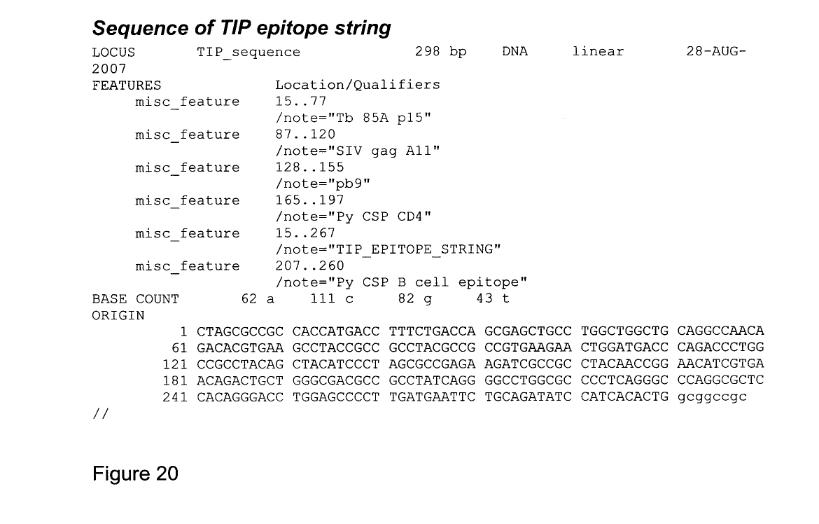

FIG. 20—is the sequence (SEQ ID NO: 1) of the TIP epitope string;

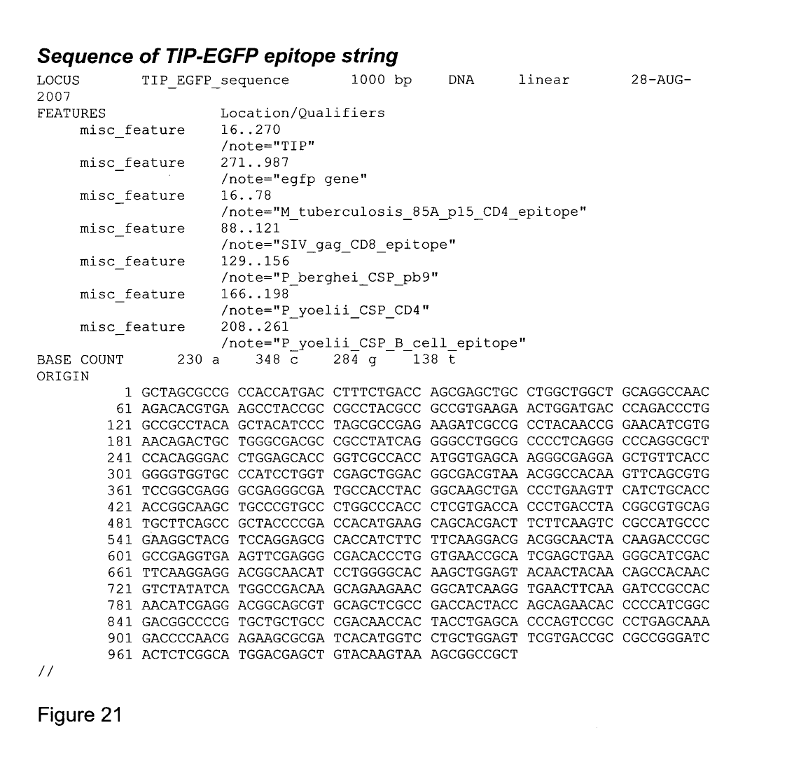

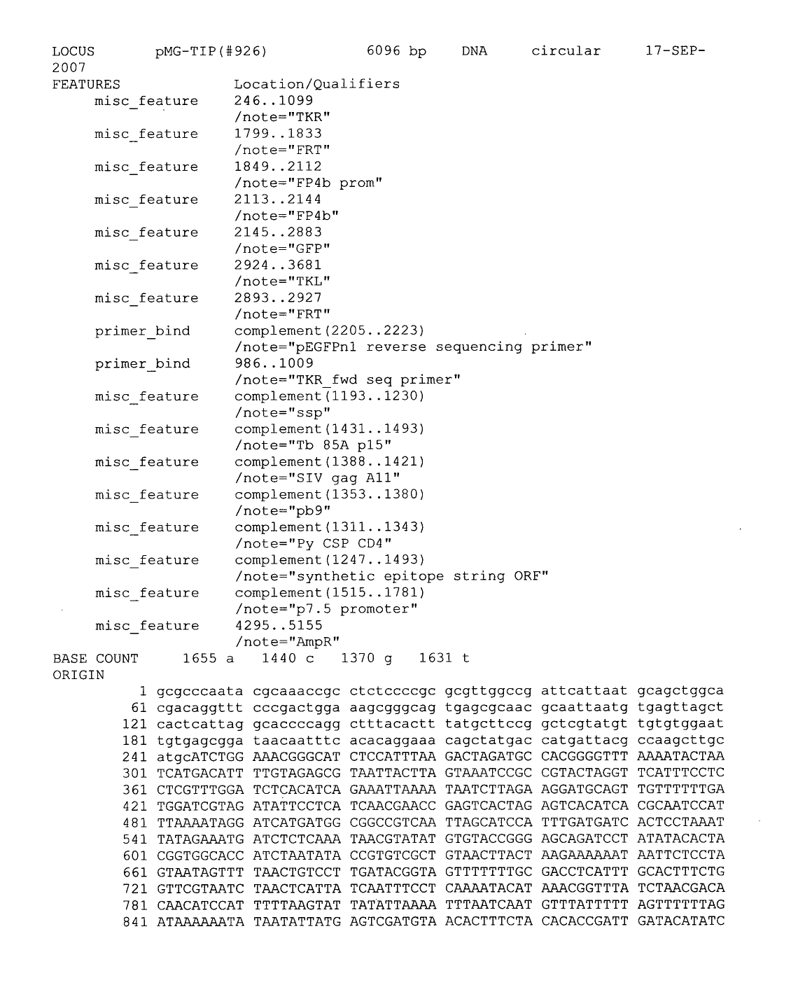

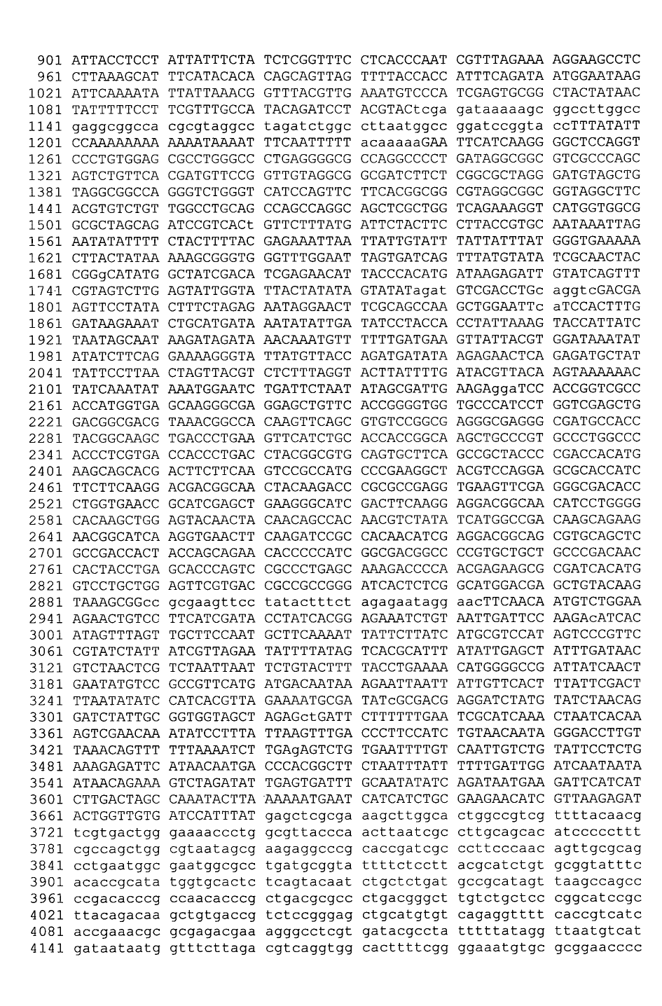

FIG. 21—is the sequence (SEQ ID NO: 2) of the TIPEGFP epitope string;

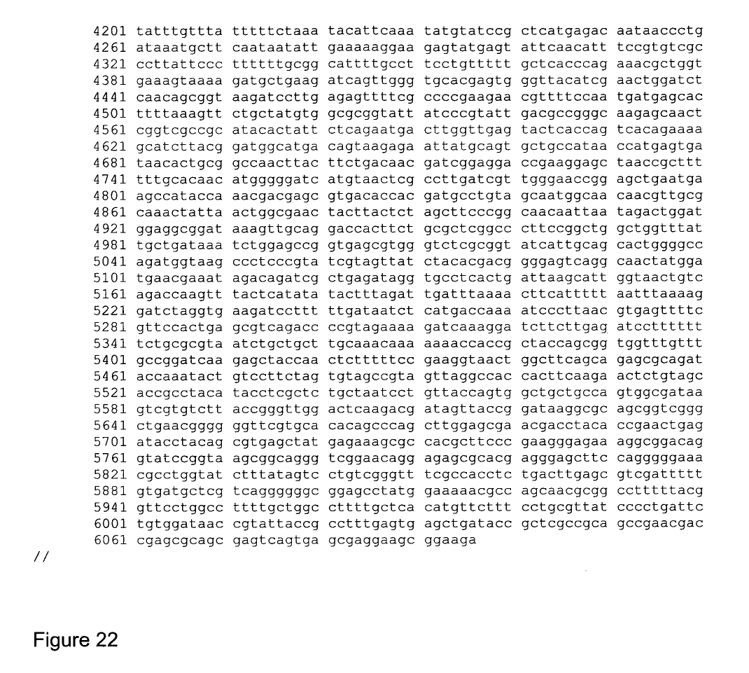

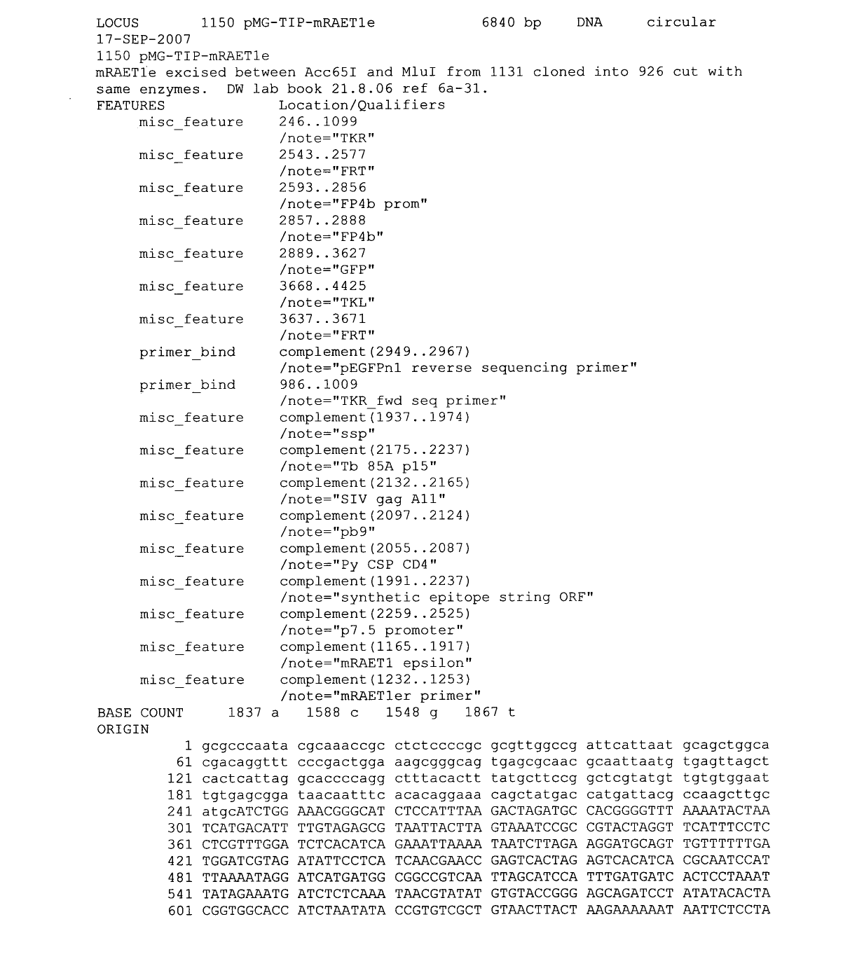

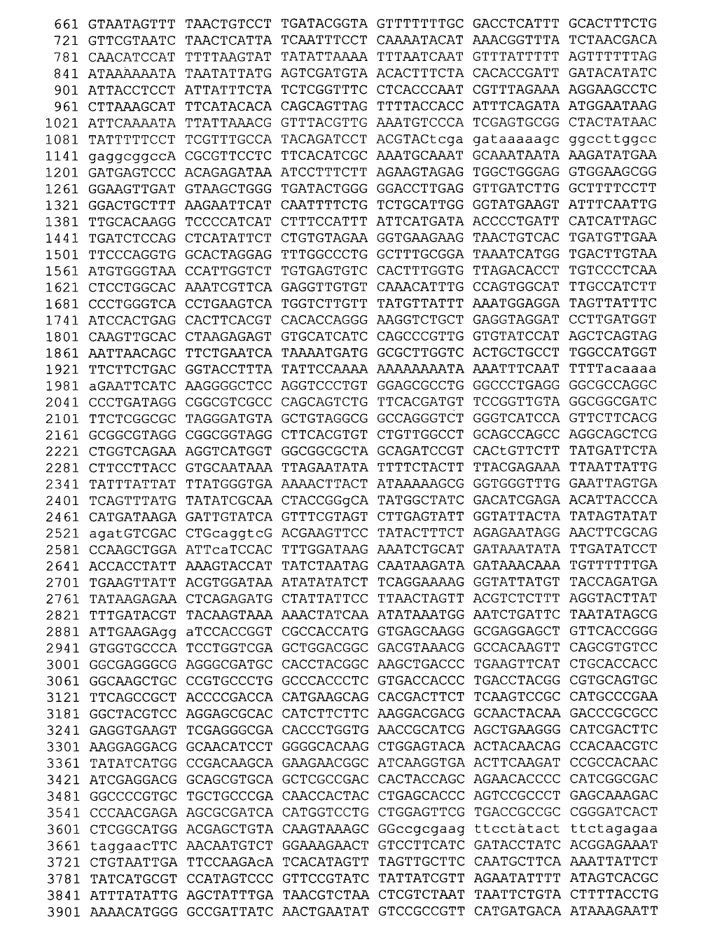

FIG. 22—is the sequence (SEQ ID NO: 3) of the shuttle vector used in the construction of the MVA-TIP control vector;

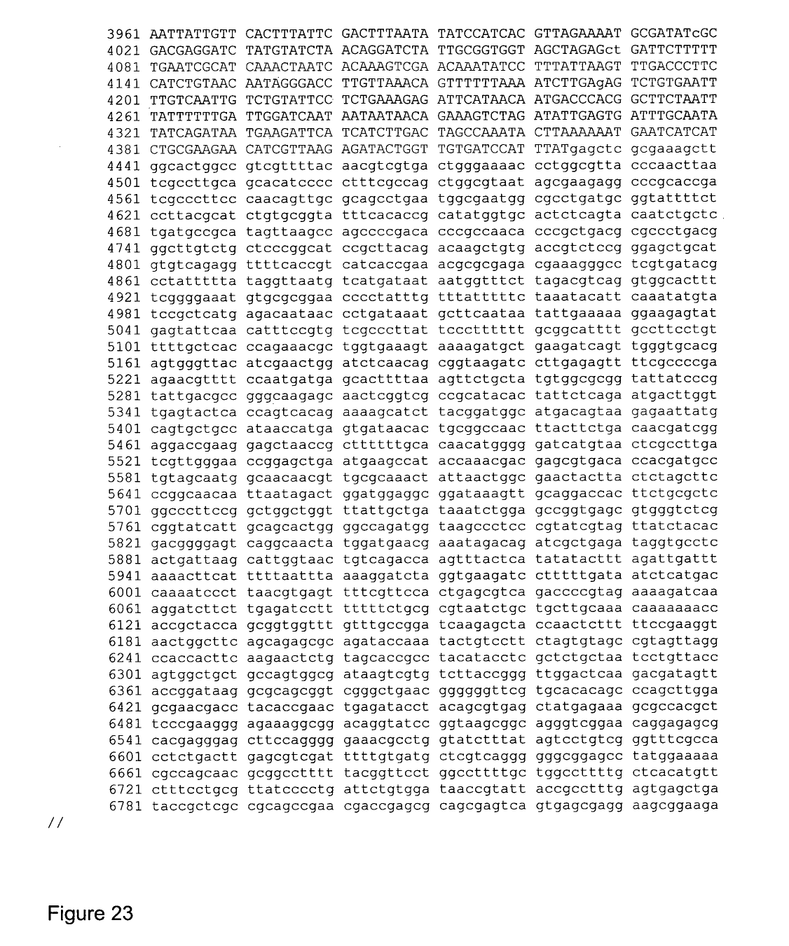

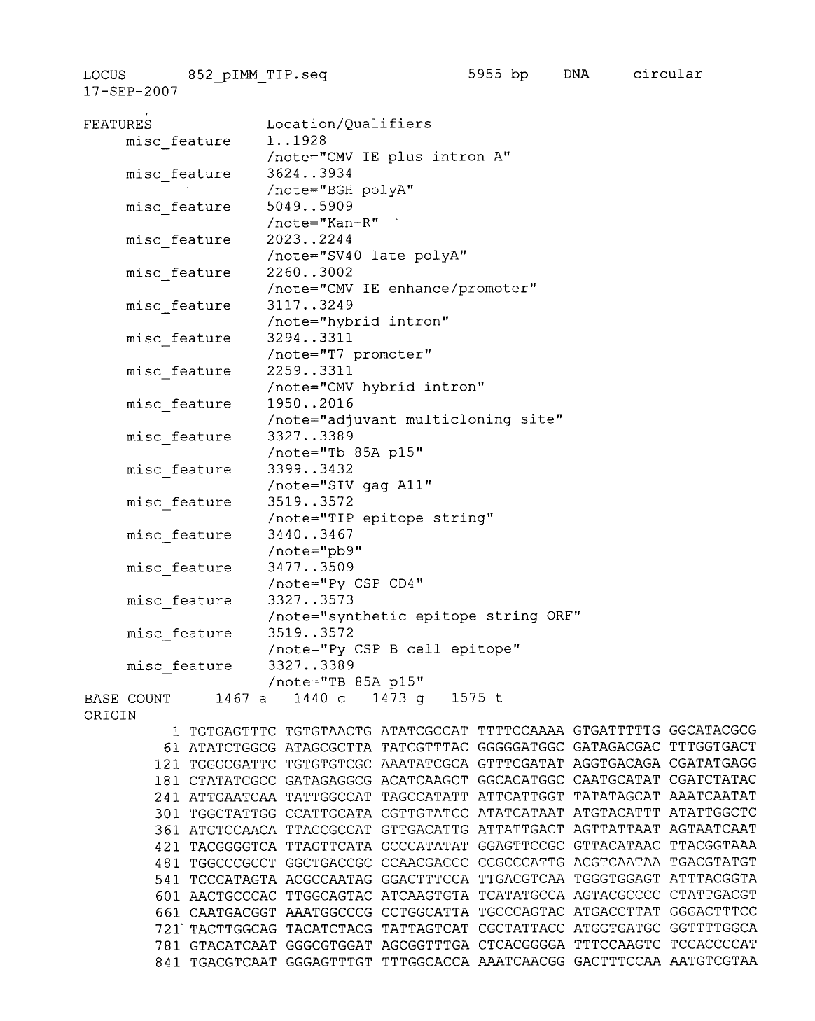

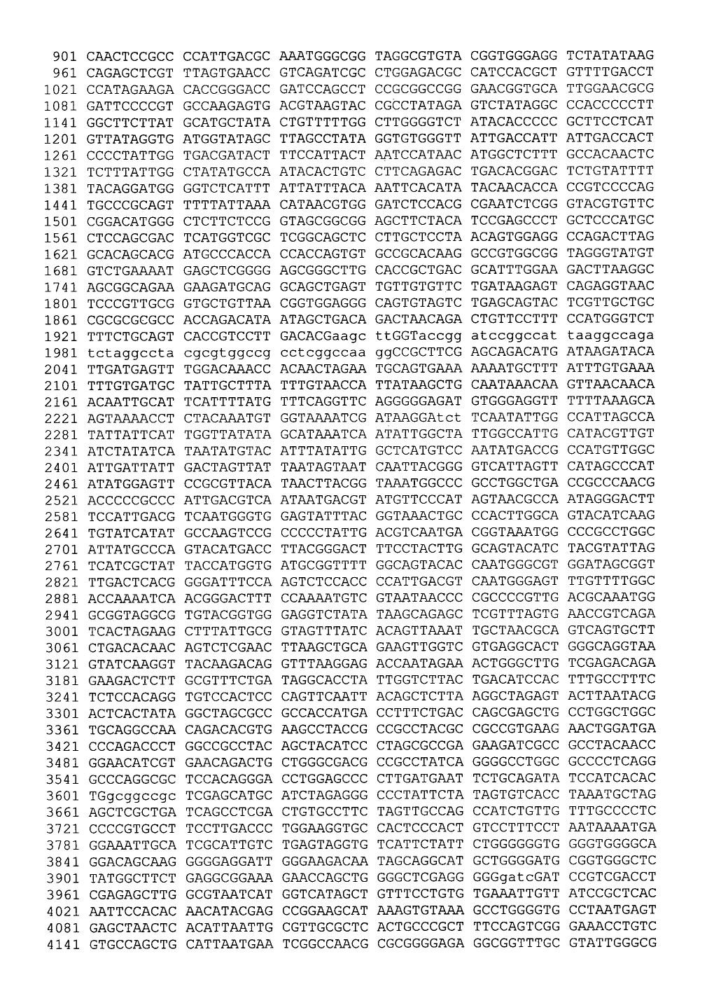

FIG. 23—is the sequence (SEQ ID NO: 4) of the shuttle vector used in the construction of the MVA-TIP-Raet1e vector;

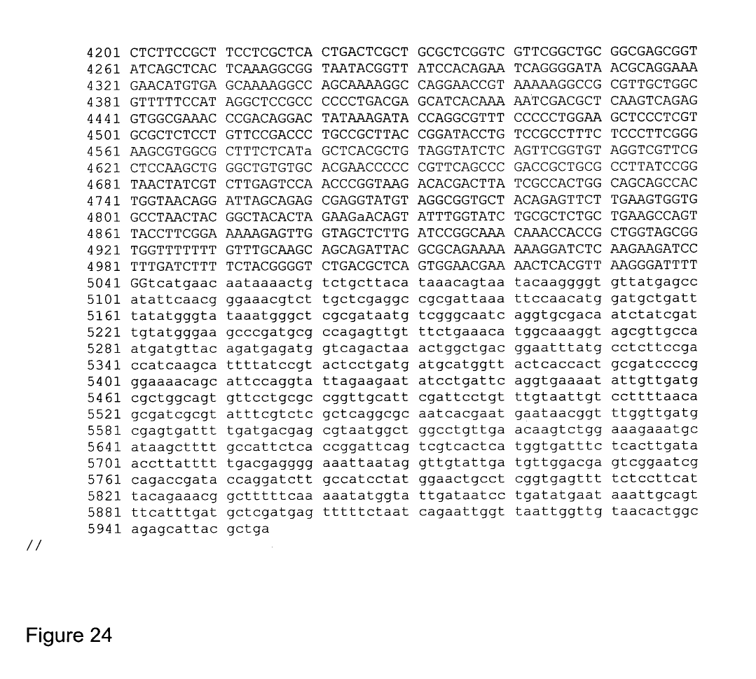

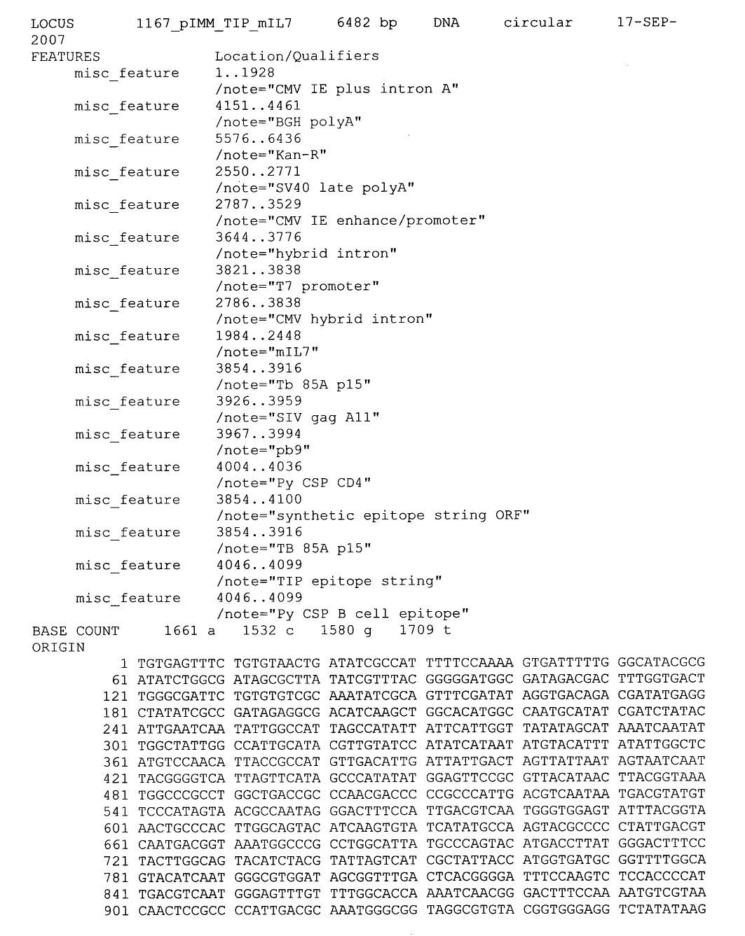

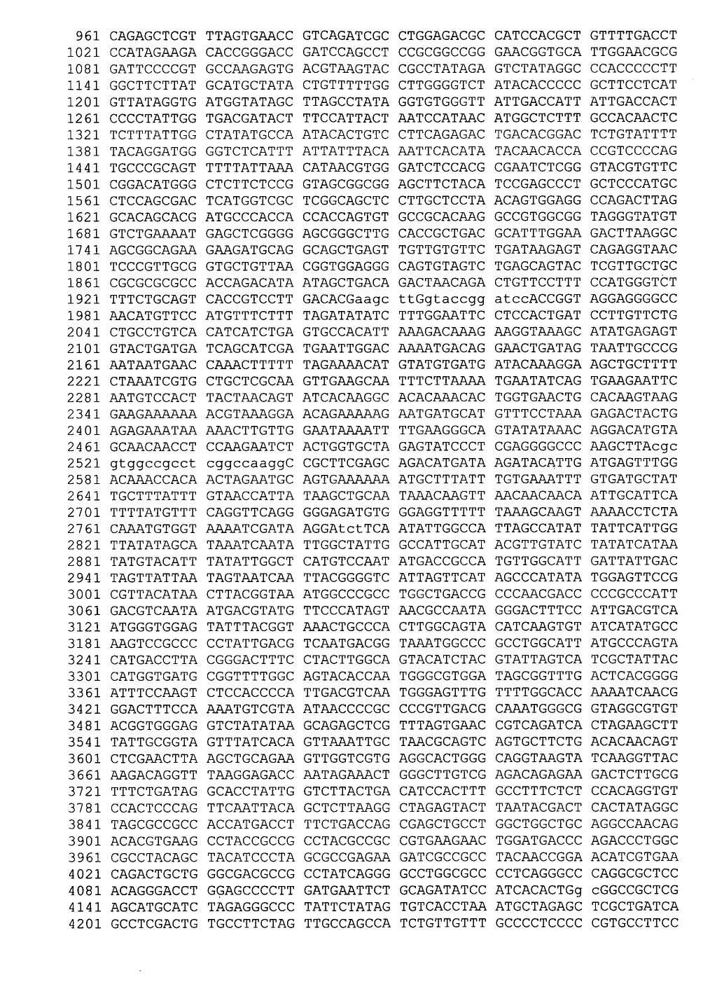

FIG. 24—is the sequence (SEQ ID NO: 5) of the shuttle vector used in the construction of the DNA-TIP control vector;

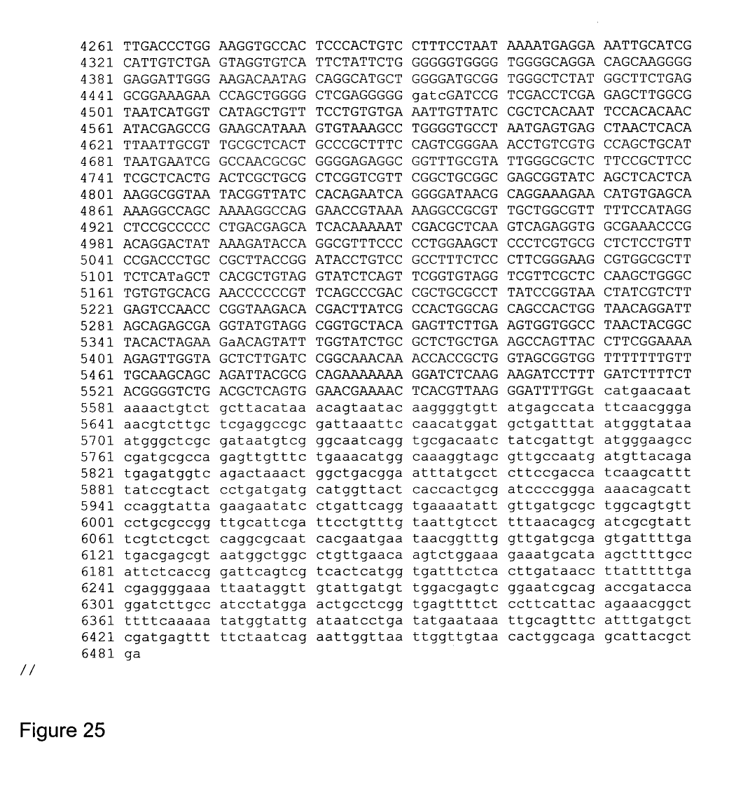

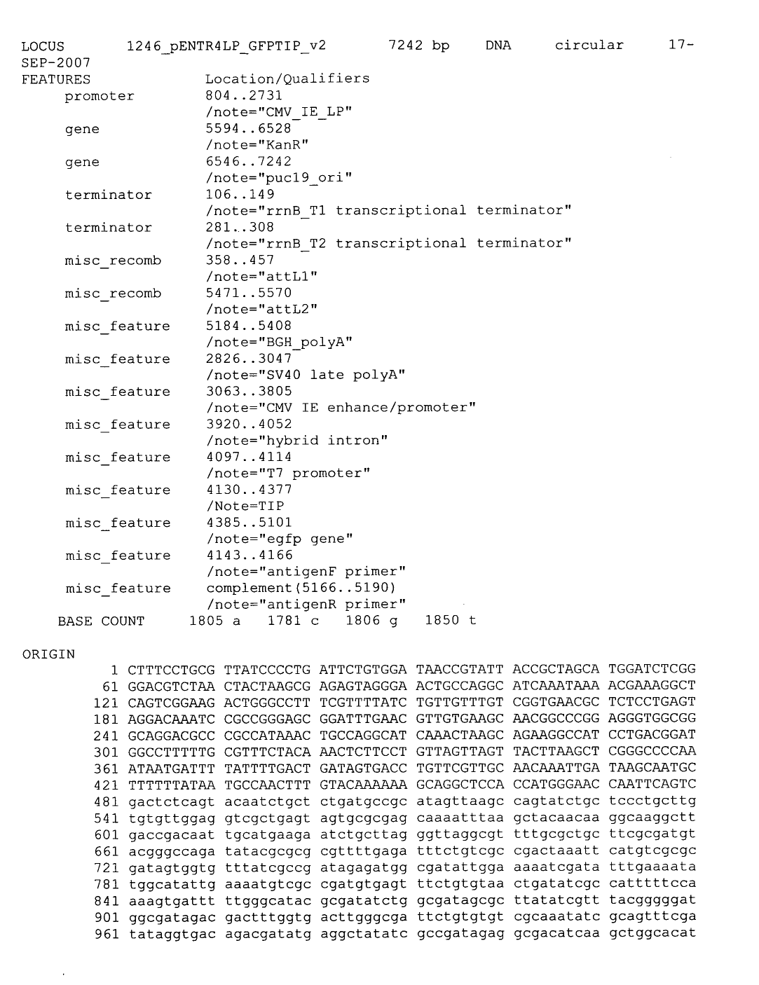

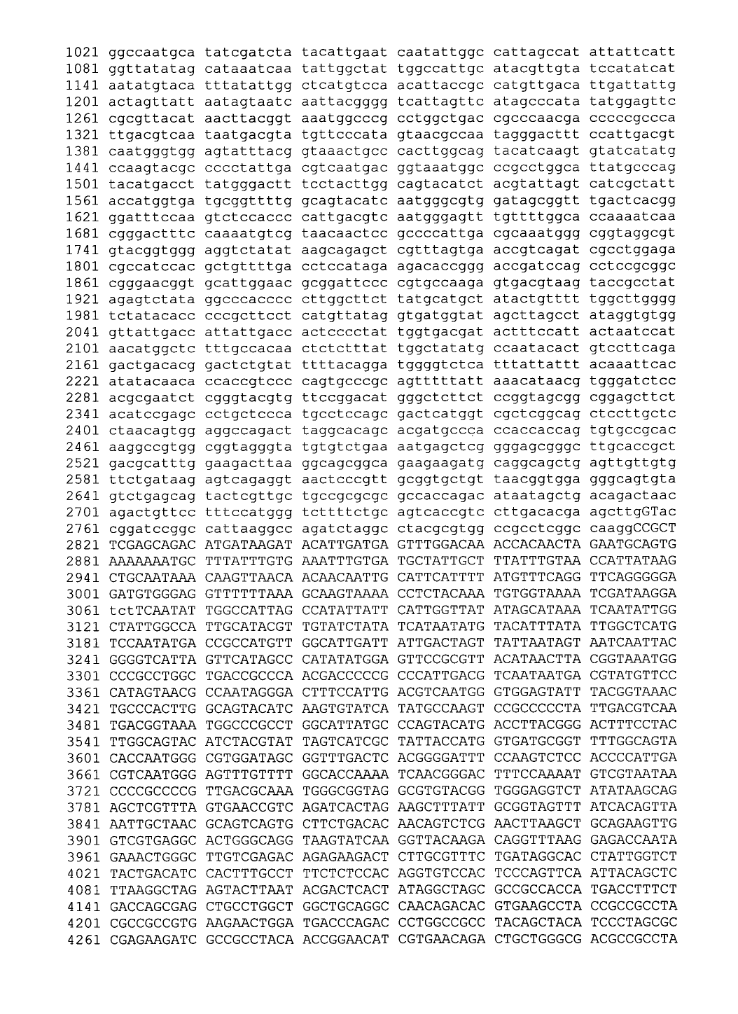

FIG. 25—is the sequence (SEQ ID NO: 6) of the shuttle vector used in the construction of the DNA-TIP-IL-7 vector;

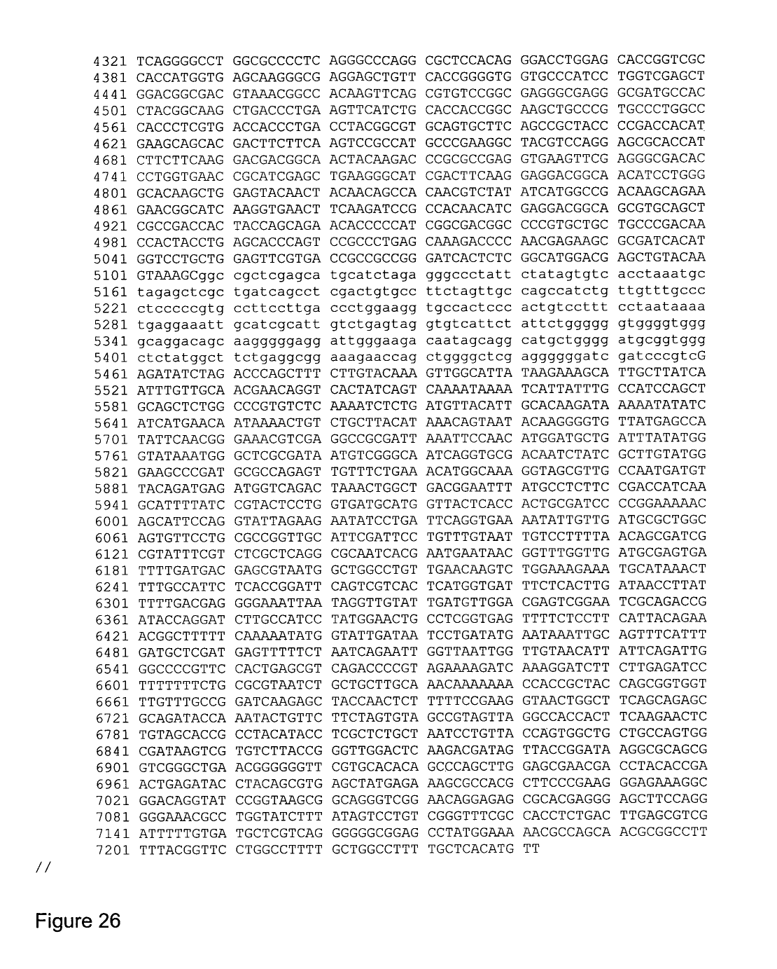

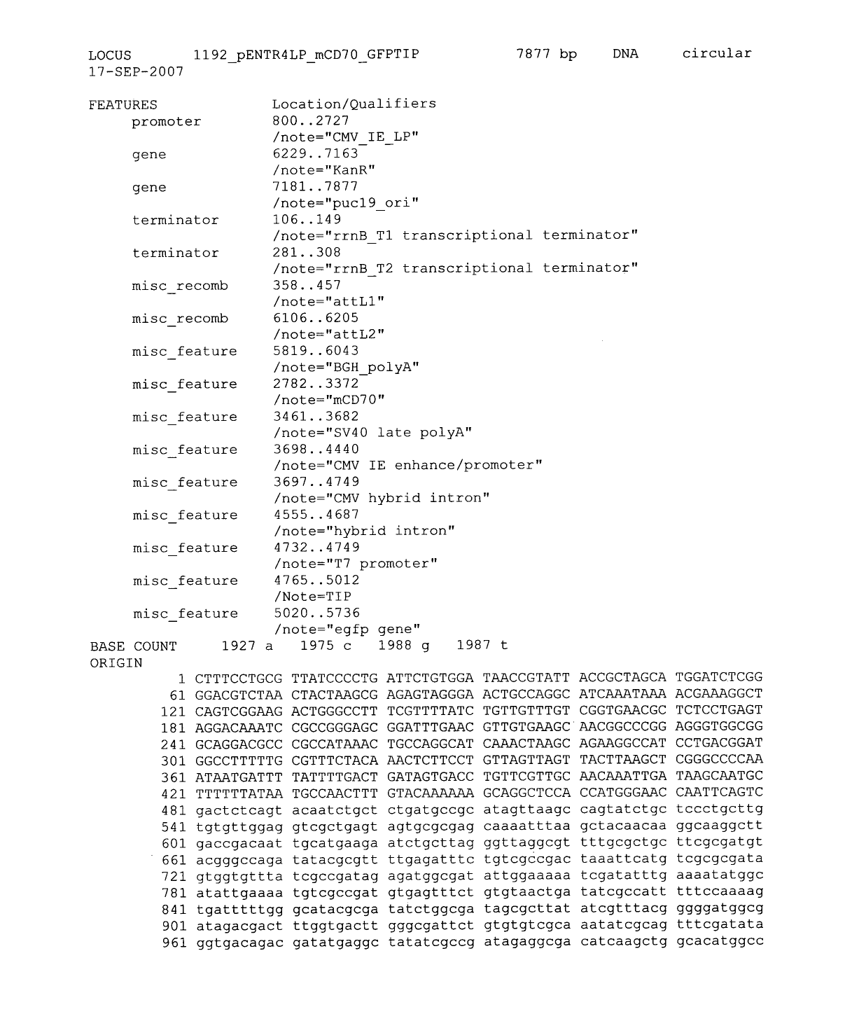

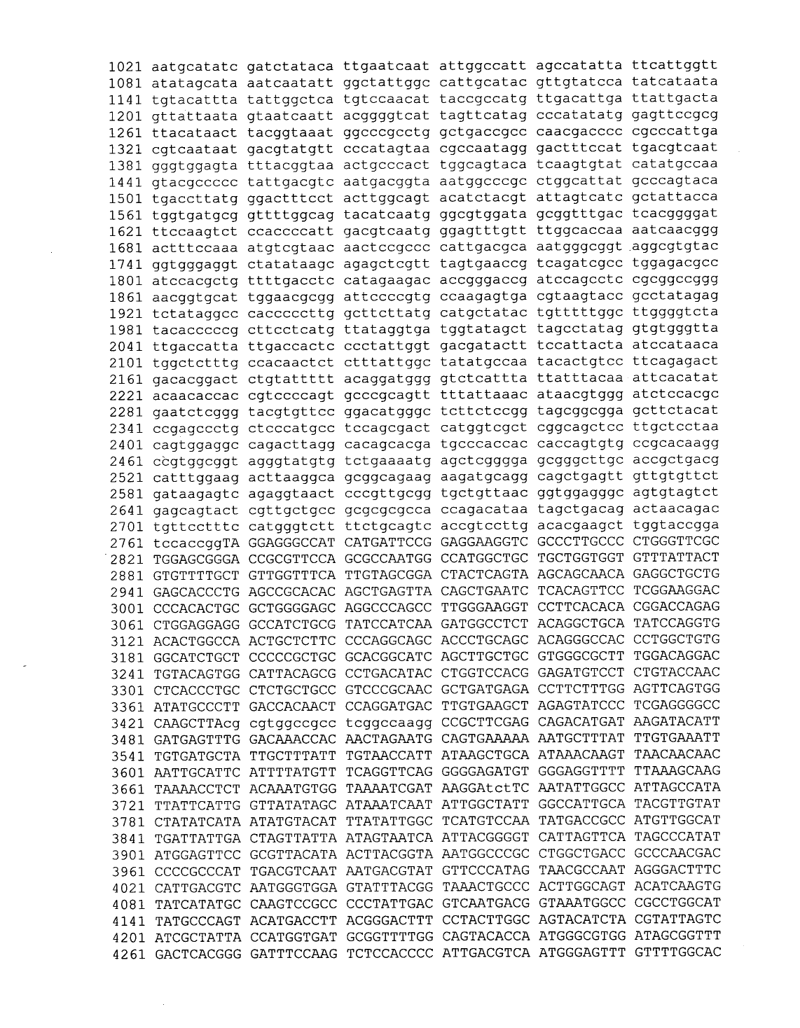

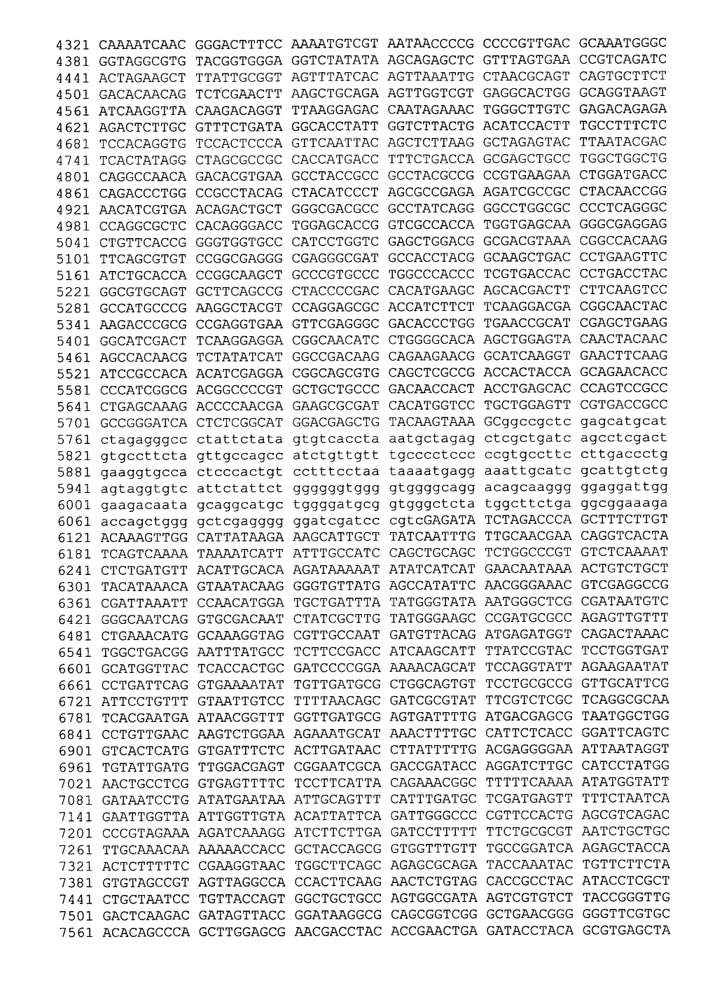

FIG. 26—is the sequence (SEQ ID NO: 7) of the shuttle vector used in the construction of the adenovirus-GFPTIP control vector;

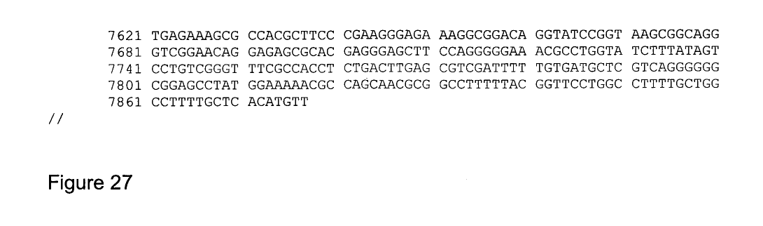

FIG. 27—is the sequence (SEQ ID NO: 8) of the shuttle vector used in the construction of the adenovirus-GFPTIP-CD70 vector.

### METHODS

### Antigens

The model antigen TIP was used in the examples described herein. TIP is an 83-amino acid polypeptide string comprising the P15 epitope from Mycobacterium tuberculosis 85A, together with epitopes from the Simian immunodeficiency virus gag protein, the CD8 Pb9 epitope, and CD4 and B cell epitopes derived from P. yoelii. Sequences of these antigens and epitopes are provided in FIG. 20. In studies of adenoviral immunogenicity, a fusion of TIP to the Aequoria victoria green fluorescent protein was used, these fusions were referred to as TIP-EGFP. The sequence of TIP-EGFP is shown in FIG. 21.

A schematic diagram of the cassette used for construction of Adenovirus Hu5 vectors, DNA vectors and MVA vectors is included in FIG. 1.

### DNA Vectors

Immunogenic DNA vectors were constructed as described herein. Except where otherwise stated, the vector used for immunization contained two CMV-promoter based expression cassettes, one expressing antigen and the other an adjuvant molecule (as illustrated in FIG. 1). Control constructs in which no molecule is present in the adjuvant site were also constructed. The vectors were assembled from previously described constructs (McConkey, S. J., et al 2003 Nat Med 9(6) 729-35) and synthetic oligonucleotides by standard methods (Sambrook, J. Molecular Cloning: A laboratory manual. 2000 Cold Spring Harbor Laboratory Press). The sequences of shuttle vectors used in the production of the DNA vectors used for immunization are shown in FIGS. 24 and 25. The skilled man will appreciate that the molecular adjuvant in FIG. 25, IL-7, can be replaced with the gene encoding any other suitable molecular adjuvant. For example, m4-1BBL (Accession no: NM 009404). mOX40L (Accession no: NM 009452), mCD70 (Accession no: NM 011617), mCD30L (Accession no: NM 009403), mRaet1ε (Accession no: NM 198193), mTRAM (Accession no: NM 173394), mTRAF6 (Accession no: NM 009424), mTAK1 (Accession no: NM_{—}172688), hNIK(

Accession no: NM 003954), mTIRAP (Accession no: NM 054096). A leading “m” indicates the mouse sequence, and a leading “h” indicates the human sequence. Either the mouse or human sequence can be used. The accession number refers to the genbank number. In producing the shuttle vectors the complementarity determining sequences encoding the molecular adjuvant was placed downstream of a cytomegalovirus immediate-early gene promoter. Construction of the shuttle vectors and the final DNA vectors used standard techniques.

### Adenoviral Vectors

Bi-cistronic expression cassettes of design similar to those used for DNA vaccination (above) were constructed and inserted into the pENTR4 vector (Invitrogen, http://www.invitrogen.com). FIG. 1 illustrates schematically the expression cassette. The pENTR4 vector produced was recombined with pAD-PL-DEST (Invitrogen, http://www.invitrogen.com) using the Gateway recombination system as recommended by the manufacturer. The sequences of shuttle vectors used in the production of the adenovirus vectors used for immunization are shown in FIGS. 26 and 27. The skilled man will appreciate that the molecular adjuvant in FIG. 27, CD70, can be replaced with the gene encoding any other suitable molecular adjuvant. For example, m4-1BBL (Accession no: NM 009404). mOX40L (Accession no: NM 009452), mIL-7 (Accession no: NM 008371), mCD30L (Accession no: NM 009403), mRaet1ε (Accession no: NM 198193), mTRAM (Accession no: NM 173394), mTRAF6 (Accession no: NM 009424), mTAK1 (Accession no: NM_{—}172688), hNIK(Accession no: NM 003954), mTIRAP (Accession no: NM 054096). A leading “m” indicates the mouse sequence, and a leading “h” indicates the human sequence. Either the mouse or human sequence can be used. The accession number refers to the genbank number. In producing the shuttle vectors the complementarity determining sequences encoding the molecular adjuvant was placed downstream of a cytomegalovirus immediate-early gene promoter. Construction of the shuttle vectors and the final adenovirus vectors used standard techniques.

### MVA Vectors

Shuttle vectors for MVA generation were constructed, the expression cassette of which is schematically illustrated in FIG. 1. The shuttle vector featured homologous recombination arms for insertion into the TK locus of Modified Vaccinia Ankara, and pox viral promoters for expressing the adjuvant and the TIP antigen were included. A separate late viral promoter was also included which directs expression GFP and can be used for selection of recombinant viruses. The sequences of shuttle vectors used in the production of the adenovirus vectors used for immunization are shown in FIGS. 22 and 23. The skilled man will appreciate that the molecular adjuvant in FIG. 23 Raet1e, can be replaced with the gene encoding any other suitable molecular adjuvant. For example, m4-1BBL (Accession no: NM 009404). mOX40L (Accession no: NM 009452), mIL-7 (Accession no: NM 008371), mCD30L (Accession no: NM 009403), mCD70 (Accession no: NM 011617), mTRAM (Accession no: NM 173394), mTRAF6 (Accession no: NM 009424), mTAK1 (Accession no: NM_{—}172688), hNIK(Accession no: NM 003954), mTIRAP (Accession no: NM 054096). A leading “m” indicates the mouse sequence, and a leading “h” indicates the human sequence. Either the mouse or human sequence can be used. The accession number refers to the genbank number. In producing the shuttle vectors the complementarity determining sequences encoding the molecular adjuvant was placed downstream of a cytomegalovirus immediate-early gene promoter. Construction of the shuttle vectors and the final MVA vectors used standard techniques.

### Virus Production

Shuttle vectors comprising the expression cassettes as illustrated in FIG. 1 and described above were constructed using standard molecular biological techniques.

Adenovirus Hu5 derivatives were constructed using the commercially available Invitrogen ViraPower system (http://www.invitrogen.com), and amplified and titred in 293A cells. Purification used Adenopure affinity chromatography (http://www.puresyn.com), or caesium chloride centrifugation. Titration was performed using absorbance-based viral particle estimation, and plaque forming unit estimation.

Modified Vaccinia Ankara (MVA) recombinants were produced by homologous recombination into the thymidine kinase locus of Modified Vaccinia Ankara, essentially as described previously (McConkey, S. J et al 2003 Nat Med 9(6) 729-35), and grown and titred on chick embryo fibroblast cells.

### Immunogenicity Assays

To assess immunogenicity of adjuvanted vectors, whether DNA, MVA or adenovirus, in mice, groups of 4-6 mice were analysed for each condition under test. Outcomes were measured using ELIspot and flow cytometry assays for responses specific to the epitopes present in TIP. In Balb/c mice, responses to Pb9, the CD8 + T cell epitope from P. berghei circumsporozoite protein (CSP), and P15, a CD4 + epitope from M. tuberculosis antigen 85A, were detected following vaccination/immunisation. To measure antigen specific IFN-γ production by ELIspot, splenocytes were stimulated with high purity Pb9 or P15 peptides at the relevant time point after immunization. The method used was essentially as described in Hanke, T et al 1198 J Gen Virol 79 (Pt 1) 83-90. Flow cytometry was used to assess the intracellular production of IFN-γ, TNF-α and IL-2 after stimulating splenocytes for 6 hours with the relevant high purity peptide. Statistical comparisons of the responses between the groups used Student t testing, or one-way ANOVA with Dunnett testing to compare adjuvanted groups with non-adjuvanted groups, as appropriate. GraphPad Prism 4 was used for these analyses (http://www.graphpad.com).

### Immunogenicity of DNA Vectors

Mice were immunized i.m. in the quadriceps muscle with DNA (either 100, 50 or 25 μg total) on days 0 and 14. On day 28 splenocyte IFN-γ ELIspot numbers were determined for Pb9 and P15 peptides. In some experiments, blood IFN-γ ELIspot assays were also performed, as described herein. In other experiments, combinations of DNA vaccines were used. In some adjuvant regimes, the fold increase in immunogenicity across experiments was compared. General linear models were generated (antigen specific cell number being determined by ELISPOT) as a function of DNA dose, experiment, and adjuvant, the latter three being categorical variables. The analysis used SPSS 14 for Windows. Maximum-likelihood estimates of fold change attributable to adjuvant from e_{Badj}, where B_{adj} is the coefficient in the linear model associated with adjuvant relative to no adjuvant, were determined. The significance of the increase from the estimate of the variance of B_{adj} was determined.

### Immunogenicity of MVA Vectors

Mice were immunised i.d. with 10⁶ PFU of MVA vectors and splenocytes were analysed at the peak of the response (7 days post-immunisation) for Pb9-specific CD8 + T cell responses by flow cytometry with intracellular staining for IFN-γ and TNF-α. Since MVA does not induce detectable responses to the P15 epitope in TIP, CD4 + responses following a single MVA vaccination could not be monitored.

### Immunogenicity of AdHu5 Vectors

The immunogenicity of adjuvanted AdHu5 vectors were studied using doses of 10⁹ and 10⁸ viral particles (vp) per mouse i.d. These doses were chosen based on an initial dose response study, in which 10⁹ vp was shown to elicit a maximal CD8 + and CD4+ T cell response, while 10⁸ induced an approximately half-maximal response. At day 14 post-immunisation, which corresponds to the peak of the response, splenocyte IFN-γ ELIspot responses were observed against Pb9 and P15 peptides.

### DNA Prime-MVA Boost

In order to assess the effect of adjuvants on the ability of MVA vectors to boost a previously primed response, mice were immunized with 50 μg of DNA vectors 14 days prior to boosting with 10⁶ pfu of MVA vector. Twelve days after the prime, Pb9-specific ELIspot responses in peripheral blood were increased relative to unadjuvanted vector in mice immunised with adjuvanted DNA.

P. berghei Challenge

Protection assays were performed essentially as described by Anderson, R. J et al 2004 J Immunol 172(5) 3094-100. 1,000 P. berghei sporozoites dissected from the salivary glands of infected mosquitoes were injected intravenously into mice 14 days following adenoviral vaccination. The mice had been vaccinated with either an adenovirus vector expressing TIP and IL-7 (Ad-TIPEGFP-IL-7) or an adenovirus vector expressing TIP bit not IL-7 (Ad-TIPEGFP). Blood smear monitoring began 3 days after injection of the sporozoites, and continued for 14 days. Time to 0.5% parasitaemia (that is, where 5% of cells studied are infected with the parasite) was used as an arbitrary endpoint. This was determined for individual mice by regression of log (parasitaemia) on time since vaccination. Kaplan-Meier survival analysis was used to determine significance of differences of times to event between groups. In experiments where multiple vaccine doses were used, analyses were stratified by dose.

### EXAMPLE 1

### Adjuvant Effects of IL-7

IL-7 is a 4-helix bundle cytokine, which interacts with a receptor consisting of IL-7Rα (CD127) and a common signalling chain, γ (CD132) (Mazzucchelli, R. and Durum S. K 2007 Nat Rev Immunol 7(2) 144-54).

IL-7 is produced from stromal cells in the bone marrow. The levels of IL-7 are rate limiting for T cells in many physiological settings (Mazzucchelli, R. and Durum S. K 2007 Nat Rev Immunol 7(2) 144-54). IL-7 is thought to have an important role under conditions of high T cell turnover, such as, in individuals with HIV. IL-7 levels are inversely correlated with lymphocyte number in HIV, and IL-7 can prevent T cell death in lymphocytes from HIV-positive patients. IL-7 is a survival factor for T cells, supporting the homeostatic turnover of memory cells, which physiologically divide at a slow rate without apparent antigenic stimulation (Mazzucchelli, R. and Durum S. K 2007 Nat Rev Immunol 7(2) 144-54).

IL-15 is a structurally related cytokine to IL-7, and shares the property of enhancing T cell survival with IL-7 (Williams M. A and Bevan M. J 2007 Annu Rev Immunol 25 171-92).

Vectors co-expressing IL-7 and an antigen (TIP) were produced and tested using the methods described above.

FIG. 11 illustrates that immunisation with an MVA vector co-expressing IL7 and the TIP antigen induces or increases the CD8 epitope (Pb9) response when compared to immunisation with an MVA vector expressing TIP but no IL7. Additionally, immunisation with IL7 as an adjuvant induced an increase in the CD4 (p15) specific response to the TIP antigen. The results in FIG. 11 demonstrate the adjuvant effect of IL7 in a prime boost immunisation regimen using a DNA vector as the prime and an MVA vector as the boost. The results illustrate that boosting with an MVA vector carrying IL7 and TIP enhances the Pb9 and P15 specific responses.

### EXAMPLE 2

### Adjuvant Effects of Co-Stimulatory Molecules

T cell proliferation following TCR-MHC engagement depends on additional signals, provided by co-stimulatory molecules present on antigen presenting cells (APC) (Watts, T. H. 2005 Annu Rev Immunol 23 23-68). The first interactions are between CD28 (T cell) and B7.1 (ligand on APC). Subsequently, engagement of a family of TNF receptor-like molecules (T cell) by membrane bound TNF-related ligands (on the APC) controls proliferation (Watts, T. H. 2005 Annu Rev Immunol 23 23-68). The TNF-related ligands are examples of co-stimulating molecules.

Although studies of mice with targeted deletions of TNF-related co-stimulatory receptors, or their ligands, have established that several of are essential for normal T cell development, additional classes of co-stimulatory molecules are increasingly recognized. These include for example NKG2D ligands (Groh, V., et al 2001 Nat Immunol 2(3) 255-60; Maasho, K., et al 2005 J Immunol 174(8) 4480-4) and ephrins (Luo, H., et al 2002 J. Clin. Invest 110(8) 1141-1150).

Here it is shown that CD70, OX40L, 41BBL, CD30L, all members of the TNF-like co-stimulatory ligand family, and an NKG2D ligand, Raet1e, enhance immunogenicity of vectored vaccines.

### Adjuvant Effects of 4-1BBL

### 4-1BBL

4-1BBL is physiologically expressed, in an inducible manner, on dendritic cells (Laderach, D. et al 2003 Cell Immunol 226(1) 37-44; Futagawa, T., et al 2002 Int Immunol 14(3) 275-86), monocytes and B cells (Langstein, J., et al 1998 J Immunol 160(5) 2488-94; Pauly, S. et al 2002 J Leukoc Biol 72(1) 35-42). Expression in the central nervous system has also been reported (Reali, C. et al 2003 J Neurosci Res 74(1) 67-73). 4-1BBL contains a cytosolic signalling domain, engagement of which induces IL-12 secretion (Laderach, D. et al 2003 Cell Immunol 226(1) 37-44) by dendritic cells. Sustained macrophage TNFα secretion also depends on 4-1BBL, which appears to form part of the TLR complex (Kang, Y. J. et al. 2007 Nat Immunol 8(6) 601-9), initiating Myd88 and TRIF independent, C/EBP dependent signaling.

### 4-1BB

The 4-1BB receptor is expressed on both CD4 and CD8 T cells (Takahashi, C. et al 1999 J Immunol 162(9) 5037-40), and is transiently upregulated after immunisation, with a peak in expression about 24 hours after immunisation (Dawicki, W. et al. 2004 J Immunol 173(10) 5944-51).

### Physiological Functions

Engagement of 4-1BB receptor prolongs T cell survival, especially of CD8 cells (Takahashi, C. et al 1999 J Immunol 162(9) 5037-40; Shuford, W. W. et al. 1997 J Exp Med 186(1) 47-55), and it enhances T cell responses during influenza and herpes virus infection (Bertram, E. M. et al. 2002 J Immunol 168(8) 3777-85; Tan, J. T. et al. 1999 J Immunol 163(9) 4859-68; Fuse, S., et al. 2007 J Immunol 178(8) 5227-36).

### Data

FIG. 6 illustrates the adjuvant effect of 4-1BBL on the immunogenicity of an MVA vector and an adenovirus vector expressing the TIP antigen. The data demonstrates that 4-1BBL enhances the CD4 and the CD8 response to the TIP antigens. The adjuvant effect of 4-1BBL is also observed in an AdHu5-MVA prime-boost regime.

### Adjuvant Effects of OX40L

### OX40

OX40 is inducible on T cells following TCR/CD28 engagement, with increased levels being observed 24-48 hrs after stimulation (Gramaglia, I. et al 1998 Journal of Immunology 161(12). 6510-6517). Engagement is associated with sustained signalling, induction of anti-apoptotic molecules (Rogers, P. R. et al 2201 Immunity 15(3) 445-455) and survivin (Song, J. et al 2005 Immunity 22(5) 621-31). Knockout data confirm that OX40 is required for long term survival of CD4 T cells (Rogers, P. R. et al 2201 Immunity 15(3) 445-455).

### OX40L

In general, levels of OX40L are low or absent on most cell types, including T cells (Serghides, L. et al 2005 J Immunol 175(10) 6368-77), in the absence of stimulation. Induction has been demonstrated on both CD4 and CD8 T cells (Rogers, P. R. et al 2001 Immunity 15(3) 445-455; Mendel, I. and E. M. Shevach 2006 Immunology 117(2) 196-204; Kondo, K., et al 2007 Hum Immunol 68(7) 563-71), B cells (Stuber, E., et al 1995 Immunity 2(5) 507-521), NK cells (Zingoni, A., et al 2004 Journal of Immunology 173(6) 3716-3724), dendritic cells (Ito, T., et al 2004 Journal of Immunology 172(7) 4253-4259), and endothelial cells (Ito, T., et al 2004 Journal of Immunology 172(7) 4253-4259; Imura, A., et al 1996 J. Exp. Med. 183(5) 2185-2195).

### Data

FIG. 7 illustrates that co-expression of OX40L and the TIP antigen by adenovirus significantly enhances both the CD4 (Pb15) and the CD8 (Pb9) immune responses to the TIP antigen, thereby demonstrating the adjuvant effect of OX40L.

### Adjuvant Effects of CD70/CD27L

CD70 is the ligand for CD27, and is also known as CD27L. It is a type II transmembrane protein.

CD70 ligand expression is normally closely regulated, however when CD27L is constitutively expressed on B cells an extensive and effective memory-like T cell pool develops (Arens, R. et al 2004 J Exp Med 199(11) 1595-605). In chronic viral infections, CD70 signalling may be relevant to outcome. Using a chronic LCMV model, it was shown that CD27 knockout, or a blockade of CD27, resulted in faster production of neutralising antibodies, and subsequent viral clearance (Matter, M. et al 2006 J Exp Med 203(9) 2145-55).

### Data

FIG. 8 illustrates that co-expression of CD70 and the TIP antigen by adenovirus vector significantly enhances the CD8 (Pb9) immune response to the TIP antigen, thereby demonstrating the adjuvant effect of CD70.

### Adjuvant Effect of CD30L

CD30L is the ligand for CD30. It is a type II transmembrane protein and its precise physiological role has yet to be elucidated.

### CD30

CD30 is expressed on B and T cells, and is induced late during in vitro activation.

### CD30L

CD30L is expressed on activated T cells, and on some B cell lines and subsets (Kennedy, M. K. et al. 2006 Immunology 118(2) 143-52). CD30L is also expressed, along with OX40L, in an IL-7 inducible manner on a CD4⁺CD3⁻ accessory cell population in the spleen, which supports T-B cooperation and memory development (Kim, M. Y. et al 2003 Immunity 18(5) 643-54; Kim, M. Y. et al 2005 J Immunol 174(11) 6686-91).

On the basis of blocking experiments and CD30 knockout mice, it is argued that the role of CD30 is control of T-cell dependent secondary antibody responses (Kennedy, M. K. et al 2006 Immunology 118(2) 143-52).

### Data

FIG. 9 illustrates that co-expression of CD30L and the TIP antigen from an adenovirus vector significantly enhances both the CD4 and the CD8 immune responses to the TIP antigen in immunised mice. Additionally, expression of CD30L by modified vaccinia Ankara (MVA) also enhances immunogenicity.

Adjuvant Efect of NKG2D ligands

NKG2D is a type II transmembrane protein expressed on NK cells, human and activated alpha-beta murine T cells, and some gamma-delta T cells. Ligands have been characterised, as reviewed by Raulet, D. H in 2003 Nat Rev Immunol 3(10) 781-90. RAET/ULBP, MICA and MICB (major histocompatibility complex class I-related molecules A and B) are identified human ligands; Rae proteins, H60 and MULT are identified as mouse ligands (Raulet, D. H. 2003 Nat Rev Immunol 3(10) 781-90).

### Data

FIGS. 2, 3, 4, and 5 illustrate the result of co-expressing of an NKG2D ligand,

Raet1e, and the TIP antigen.

Immunogenicity of an MVA vector

FIG. 2 illustrates that immunisation with an MVA vector co-expressing Raet1e and the TIP antigens induces a statistically significant increase in the CD8 epitope (Pb9) specific response when compared to immunisation with an MVA vector expressing TIP but no Raet1e. The increase ranged from 1.4 to 1.8-fold in two independent experiments.

### Immunogenicity of an Adenovirus Vector

FIG. 2 illustrates that immunisation with an AdHu5 (Adenovirus) vector co-expressing Raet1e and the TIP antigens induces a significant increase in the CD8 Pb9-specific response observed by ELIspot analysis, compared to immunisation with an AdHu5 vector expressing TIP but no Raet1e.

FIG. 3 illustrates that for the P15 CD4 epitope, statistically significant enhancements in immunogenicity were observed for all Raet1e adjuvanted vectors.

To assess the effect of IL-7 on the protective effect of an adenovirus based vaccine using the TIP antigens, mice were immunized with two doses of Ad-TIPEGFP-Raet1e or control virus (without Raet1e). Enhanced immunogenicity with Raet1e adjuvanted viruses was observed in an interferon-gamma ELIspot performed on blood analysed 2 weeks after immunisation (FIG. 4), which shows the immunogenicity (above) and the number of animals protected/number of animals tested.

This protective effect was associated with a significantly greater protection against experimental P berghei challenge (FIG. 5). FIG. 5 illustrates that in mice immunised with 1×10⁹ adenovirus expressing the TIP antigens and the adjuvant Raet1e a much greater number survived a P berghei challenge than if immunised with an adenovirus vector encoding the TIP antigens but no Raet1e adjuvant. More specifically, 7 days after immunisation with antigen and adjuvant about 50% of mice were still alive, whereas 80% of mice immunised with an adenovirus expressing the TIP antigens, and no Raet1e, were dead by 7 days. This demonstrates the efficacy of the vaccine in conferring protection on an animal.

### DNA Prime Boost Vaccine

The adjuvant effect of Raet1e was evaluated in a prime boost vaccination regimen using a DNA vector or an adenovirus vector as the prime and an MVA vector as the boost. The results in FIG. 3 illustrate the boosting with an MVA vector carrying Raet1e and TIP enhances the Pb9 specific response when compared to boosting with an MVA vector expressing TIP but no Raet1e. Additionally, when boosting with an MVA vector expressing Raet1e, expression of Raet1e in both the DNA prime and an MVA boost led to a significant enhancement of the Pb9-specific CD8 +response (FIG. 2) and the CD430 specific P15 response (FIG. 3), relative to controls. Raet1e is therefore capable of acting as an adjuvant both during DNA priming and MVA boosting.

### EXAMPLE 3

### Adjuvant Effects of TLR Signalling Pathway Components DNA Vector

The ability of combinations of TLR signalling pathway components to enhance the immunogenicity of DNA vaccines was studied. Mice were injected intramuscularly (according to the regime in the methods section) with 25, 50 or 100 mcg of plasmid DNA encoding the TIP antigens, a control plasmid did not encode an adjuvant and the test plasmid encoded a TLR signalling pathway component in addition to the TIP antigens. Initially, equal amounts of two plasmids of the design shown in FIG. 1 were mixed together and injected. One plasmid expressed one TLR signalling pathway component, the other a second TLR signalling pathway component. Both expressed the TIP antigen. It was observed that mixtures in which TAK1 is expressed by one plasmid and TRAM by other consistently showed a small and highly significant increase in immunogenicity, as assessed by Pb9 gamma-interferon ELIspot in blood the spleen (FIGS. 12A and 12B). Estimates of increase in immunogenicity were 1.5 in blood (p<0.001) and 1.4 in spleen (p<0.001).

A second regime was also studied in which TAK1 and TRAM were co-expressed from the same plasmid, and co-injected with an antigen-expressing plasmid, pSG2.TIP, according the regime illustrated in FIG. 13. An adjuvant effect was also observed in the spleen when the combination of adjuvants were administered in this way (FIG. 14).

### Adenovirus Vector

Using a system similar to that used to investigate co-stimulatory molecule function, the effect of NIK, TIRAP and TRAM as adenoviral adjuvants was studied (FIGS. 15 and 19A). The results show that NIK expression increases the immune response to the pathogen derived epitope Pb9. The results in FIG. 19A illustrate that TRAM increases the immune response to TIP-EGFP.

To assess the capacity of some other TLR pathways components to adjuvant TIP responses were assessed for an adenoviral vector (FIG. 19A) showing that TRAM again increased the response to Pb9 in TIP-EGFP. However, when expressed by plasmid DNA vectors clearly neither NIK nor TAK1 enhanced immunogenicity (FIG. 19B).

To assess whether vector mixtures, with one vector expressing the adjuvant and the other vector the antigen could be used to enhance immune responses to the antigen the experiment of FIG. 19C was conducted. Here a mixture of one adenovirus expressing TIP-EGFP and TRAM was used with another adenovirus expressing the MSP1 Plasmodium falciparum blood-stage antigen MSP1 which contains both CD4 and CD8 epitopes in C57/BL6 mice. (The four CD8 epitopes are, or are contained within, the following peptide sequences which were used in the assay: peptide M86=IPYKDLTSSNYVVKD; peptide M10032 INDKQGENEKYLPFL; peptide M140 =TKPDSYPLFDGIFCS; peptide M149=YRSLKKQIEKNIFTF. The one CD4 T cell epitope is: peptide M188=DKIDLFKNPYDFEAI.) The TIP epitope string contains the SIV CD8 T cell epitope for this mouse strain. The results (FIG. 19C) show that the TRAM adjuvant enhanced the SIV CD8 T cell response in cis (ie both this epitope and the TRAM are expressed from the same vector) but remarkably also clearly enhanced the MSP1 T cell responses in trans (ie the MSP1 is expressed from a different vector to that expressing the TRAM adjuvant), to both the single CD4 epitope tested and the mixture of 4 CD8 epitopes tested. This is surprising because it has been unclear whether a mixture of vectors would lead to many cells being co-infected by both. Also it would be expected that such co-infection would be required for the TRAM adjuvant to act intracellularly to enhance immune responses to the MSP1 antigen.

1-101. (canceled)

102. An immunogenic composition comprising a first vector encoding one or more target antigens and a second vector encoding one or more co-stimulatory molecules.

103. The composition of claim 102, wherein the first and second vectors are selected from the group comprising a DNA vector, a pox viral vector, a vaccinia derived non-replication competent viral vector and an adenoviral vector.

104. The composition of claim 103, wherein the first and second vectors are either adenoviral or MVA vectors.

105. The composition of claim 103, wherein the first and second vectors are not fowl pox virus or replication-competent vaccinia virus vectors.

106. The composition of claim 102, wherein one co-stimulatory molecule is encoded on a first vector and one antigen is encoded on a second vector.

107. The composition of claim 102, wherein the first and second vectors are the same type of vector.

108. The composition of claim 102, wherein the first and second vectors are different types of vector.

109. An immunogenic composition comprising an adenoviral or replication deficient orthopox viral vector encoding one or more target antigens and one or more co-stimulatory molecules.

110. The composition of claim 109, wherein the immunogenic composition comprises a replication deficient orthopox viral vector that is MVA.

111. The composition of claim 102, wherein the one or more co-stimulatory molecules are selected from the group comprising 4-1BBL, OX40L, CD70 and CD30.

112. The composition of claim 102, wherein the one or more vectors also encode a further molecular adjuvant that is not a co-stimulatory molecule.

113. The composition of claim 112, wherein the further molecular adjuvant is selected from the group comprising a TLR signaling pathway component, IL7 and IL15.

114. The composition of claim 102, wherein the one or more target antigen is derived from a pathogen or a cancer.

115. The composition of claim 102, wherein the one or more co-stimulatory molecules and the one or more target antigens are operably linked to one or more promoters.

116. The composition of claim 109, wherein the one or more co-stimulatory molecules are selected from the group comprising 4-1BBL, OX40L, CD70 and CD30.

117. The composition of claim 109, wherein the one or more vectors also encode a further molecular adjuvant that is not a co-stimulatory molecule.

118. The composition of claim 117, wherein the further molecular adjuvant is selected from the group comprising a TLR signaling pathway component, IL7 and IL15.

119. The composition of claim 109, wherein the one or more target antigen is derived from a pathogen or a cancer.

120. The composition of claim 109, wherein the one or more co-stimulatory molecules and the one or more target antigens are operably linked to one or more promoters.
